(19) United States
(12) Patent Application Publication
(10) Pub. No.: US 2024/0366464 A1
(43) Pub. Date: Nov. 7, 2024

(54) ARM THERAPEUTIC COMPRESSION SYSTEM, APPARATUS & METHODS OF USE

(71) Applicant: Sun Scientific, Inc., Dobbs Ferry, NY (US)

(72) Inventors: Sundaram Ravikumar, Briarcliff Manor, NY (US); Guy Orborne, Trumbull, CT (US); Vikram RAVIKUMAR, New York, NY (US); HARRY ALLAN ALWARD, Shelton, CT (US)

(73) Assignee: Sun Scientific, Inc., Dobbs Ferry, NY (US)

(21) Appl. No.: 18/285,853

(22) PCT Filed: Apr. 8, 2022
(86) PCT No.: PCT/US22/24121

§ 371 Date: Oct. 5, 2023

Related U.S. Application Data
(60) Provisional application No. 63/171,541, filed on Apr. 6, 2021.

(51) Int. Cl. A61H 9/00; A61F 13/00; A61F 13/64

(52) U.S. Cl. CPC A61H 9/0092; A61F 13/64; A61H 9/0057; A61F 2013/0028; A61H 2201/0176; A61H 2201/5071; A61H 2205/06

(57) ABSTRACT

The invention is directed to a therapeutic compression system including an apparatus to reduce bleeding and assist recovery post-dialysis at a needle access site. The therapeutic compression apparatus includes a bladder having a transparent portion to visualize the needle access site as well as an elongated tab as part of the hook and loop portion for self-application by the user or patient. The therapeutic compression apparatus includes compression not just on the needle access site but alongside the site for increased and faster recovery. The transparent portion of the bladder may be in the shape of a circle, oval, kidney, elongated kidney, square, rectangle, “U”, “C”, and other known shapes depending on the needle access site(s) of the patient during and after dialysis treatment.

### RELATED APPLICATIONS

The present application claim priority from PCT Application Serial No. PCT/US22/024121 based on provisional application Ser. No. 63/171,541 filed Apr. 6, 2021, and entitled “Arm Therapeutic Compression System, Apparatus and Methods of Use”, the entire contents of which are hereby incorporated herein by reference for all purposes.

### BACKGROUND OF THE INVENTION

### I. Field of the Invention

The subject invention is directed generally to a system for applying compression to an arm, and more particularly, to a system for applying compression to the lower or upper arm of an individual in conjunction with the post-treatment of dialysis to assist in controlling bleeding at the needle site on the arm and swelling on the arm. The system includes at least one therapeutic compression apparatus, such as a wrap having a partially transparent bladder and an inflation means for the bladder with the apparatus capable of applying compression to a limb such as the full arm, lower arm or upper arm and a pump connected to the inflation means.

### II. Background of the Related Art

Hemodialysis is the treatment for kidney problems where blood is put through a filter outside a patient's body, cleaned, and then returned to the patient in essence performing the function of the kidneys. It is done either at a hospital, dialysis facility or at home, though most typically at a dialysis facility. The patient generally has to travel to a hospital or a dialysis facility three days a week and undergoes the treatment for 3-7 hours per day. The number of patients being treated at home for dialysis is increasing and such patients may have treatment for 4-7 days a week. More than 661,000 Americans alone have kidney failure and of those about 468,000 are treated through hemodialysis.

Treatment of dialysis typically involves the use of two needle sites, one with the blood being removed from the patient to run through the dialysis machine to “clean” the blood and then a second needle site where the blood is returned into the patient. Generally, these two needle sites are either together on the forearm or upper arm, spaced apart by a couple of inches or more.

At some point the patient may have to undergo a minor surgely to insert or create a fistula to provide direct access to the bloodstream. The fistula may include an artely and vein joined together under the skin in an arm which can then be used for hemodialysis for years. Another option is a graft wherein a plastic tube is used to join an artery and vein under the skin. However, a graft does not last as long and will need to be redone and there is a higher risk of infection and bleeding after the hemodialysis treatment.

Whether a puncture site for a needle or a fistula or graft, each of these access sites have a tendency to bleed immediately after the treatment when the needle is removed, as well as a day or so afterward. The access site has to be observed for bleeding, redness, pus, and swelling. The current way to deal with bleeding immediately after the hemodialysis treatment is to place a piece of gauze on the site and press with the fingers for 10 minutes. This pressure is typically applied by a trained professional medical staff person such as a dialysis technician or nurse at the dialysis facility taking time away from other tasks by the professional. It is recommended to apply firm pressure over the exact bleeding site, use gauze and two fingers or thumb. It is not recommended to use a towel absorbent cloth, as this will make it difficult to see where the fistula is bleeding from. The medical staff such as the dialysis technician or nurse is best suited to apply pressure and control bleeding versus the patient himself or herself.

In 2017, the United States government paid, on average, $248 per dialysis treatment compared to $1041 per treatment with private insurance. In 2018 US Medicare spent $81 billion for people with kidney disease (National Kidney, 2020). One of the complications that occurs with hemodialysis is bleeding control from the access site which is a major complication following each dialysis session. Bleeding control is typically accomplished through manual control by the patient or the trained professional medical staff such as a dialysis technician or nurse. A piece of gauze is placed on the needle access site and then held my fingers or a thumb. Certain aids such as clamps with chitosan dressings may also be applied however they can be painful and tend to slip causing further bleeding and discomfort as well as swelling. The clamps have a very high pressure which may cause bruising and further swelling and pain to the patient. In contrast the manual control lacks consistent pressure as a human being is applying the pressure or compression, is time consuming for the medical staff and may be a waste of precious time for the medical staff who instead could be performing other procedures.

Further, bleeding may occur in between dialysis sessions at the needle access site requiring visits by the patient to urgent care or even an emergency room at a cost of approximately $10,000 per visit. Moreover, infection can occur at the needle access site if the bleeding is uncontrolled and continues either immediately after the hemodialysis treatment or at home or work in between treatment sessions. It is estimated that there are nearly 21,000 annual complications in regard to hemodialysis therapy and vascular access complications account for 16-25% of hospital admissions. Thus, a need exists for an apparatus to control bleeding both immediately after the hemodialysis treatment and in between treatment sessions for the patient with at home or at work or school. In certain cases, death can result from dialysis vascular access hemorrhaging and it is estimated that dialysis patients may die of fatal hemorrhaging approximately 0.5% though records of death from these causes are likely to be under reported.

The further need exists for such an apparatus that can be applied by the patient himself or herself without medical staff or a professional involved. If hemodialysis treatment occurs at home without professional medical staff present the potential for bleeding and swelling increases. Thus a need exists for an apparatus to reduce or stop bleeding immediately after the hemodialysis treatment as well as reduce swelling and bleeding in between treatment sessions which apparatus or system can be applied by the patient themselves without a professional medical staff person such as a dialysis technician or a nurse. It is estimated that the current manual compression immediately after a hemodialysis treatment (a dialysis technician or nurse placing gauze on the needle access site and pressing with his/her fingers or thumb for 15 minutes) results in about 39 hours of bleeding control per patient per year based on 15 minutes with patients attending 3 weekly sessions per year, equaling about 156 sessions per year and 39 hours of bleeding control. Therefore the time cost based on salary of the professional medical staff engaged in this manual compression to control bleeding may cost about $1,365 per/patient based on an annual salary of a nurse in the range of about $80,000.

If compression bandaging is used to attempt to control bleeding after a hemodialysis treatment or in between treatment sessions, current known compression bandaging, also called wrapping, involves the application of several layers of padding and sho11-stretch bandages to the involved areas. Soft-stretch bandages are preferred over long-stretch bandages (such as those normally used to treat sprains), as the long-stretch bandages cannot produce the proper therapeutic tension necessary to safely reduce lymphedema or swelling and may in fact end up producing a tourniquet effect. The short-stretch bandages enhance the pumping action of the lymph vessels by providing increased resistance for them to push against. This encourages lymphatic flow and helps to soften fluid-swollen areas of the needle site post-dialysis treatment. However, compression wraps applied to the needle site post-dialysis treatment have a problem of obscuring the needle site to check for bleeding. Thus, a need exists for a compression wrap applied to the needle site after a dialysis treatment which provides visual access to the needle site to check for bleeding.

Known methods for swelling and lymphedema treatment, like compression bandaging, have several disadvantages. The bandaging is time consuming and the effectiveness is limited to the skill of the provider. In some instances, bandages can be applied too tightly or too loosely and may slip from their intended position, decreasing their effectiveness, such as on the ann. When this occurs, bandages must be taken off and reapplied, further increasing the time of application and decreasing the consistency of application of the therapy.

The effectiveness of many of the current compression therapies is limited by the application of current products. Because current compression therapy is done either with manual wraps or electromechanical systems, they require either a skilled medical processional to apply and/or the need for the patient to be stationary for extended periods of time. Although stockings and/or bandages can be worn by patients and self-administered, they are very difficult for the patient to put on and pose a challenge for unskilled medical professionals to apply consistently and effectively.

Thus, a need exists for an apparatus and method of use to treat bleeding after hemodialysis treatment at the needle site or access site. This apparatus needs to be applied by a patient without the direction of a medical trained professional such as a dialysis technician or nurse to free up the medical staff's time and use. A need exists for an apparatus and method of use to visually observe the needle access site for any bleeding while applying compression and pressure to control the bleeding and reduce or eliminate swelling at the site and surrounding limb, such as an arm. The patient's mind, as well as medical professionals, will be at ease with visualization of the needle access site post-dialysis treatment to monitor bleeding and bruising.

Again, the current compression therapy is done either with a dialysis technician or nurse placing two fingers or a thumb on gauze placed over the access or needle site for 10-15 minutes, then placing a bandage over the site and sending the patient home. A need exists for a new system to treat bleeding post-dialysis treatment to reduce swelling and bleeding while having visualization of the needle access site. A further need exists for compression not just on the needle site but also on the area surrounding the needle site to assist in recovery and stopping the bleeding at the needle site once the needle is removed. Compression on the arm localized around the needle site and extending around the circumference of the side or even entire arm will assist in recovery and may decrease bleeding and bruising at the needle site as well as having other advantages known for compression therapy such as sore muscles and skin tenderness and other advantages.

Certain compression devices are known which may include an inflation means capable of providing constant static pressure for a period of time as well as providing intermittent varying pressure for a prior of time, but the currently known devices are bulky as the inflation means or pump is an integral part of the wrap mechanism. A need exists for an apparatus with a constant compression or pressure level to control bleeding and swelling at a needle access site after hemodialysis treatment and in between treatment sessions. A further need exists for such a compression devices or apparatus to include a check valve to prevent both over-inflation and to also seal and prevent deflation while the user engages in activities or rests in place. A need exists for a therapeutic compression system including a compression apparatus having an integrated valve to maintain the level of compression and prevent deflation of a bladder, and preferably a self-sealing valve. A need exists for a therapeutic compression system including a relief valve for immediate deflation of the compression or pressure within the bladder.

A need exists for a system in which a compression garment includes a universal connector to a variety of pumps. Thus, the user could interchange between an intermittent pneumatic pressure pump, for instance when seated, and then change to a set pressure pump for instance when walking. The user could connect to a hand pump, an electrical pump, a mechanical pump and any other type of conventional or inventive pumps without any additional converted needed. The various type of pumps are not limited to the two listed above but could be any type of pump with a universal connector.

A further need exists for a system including an inflation means which is less bulky than known systems. The user can thus return to life activities sooner than with known compression systems, prophylaxis systems and other treatment systems which limit the user's ambulation both within and outside the home due to power constraints (electrical, mechanical, battery, manual, etc.) on the system. The patient therefore has the advantage of mobility and being able to move around while the compression is being applied to the needle site area.

The apparatuses, methods, assemblies and systems of the subject invention provide benefits and advantages that may overcome a number of problems with respect to known compression technologies, particularly the problems that arise due to the difficulty of applying current manual compression or current compression wrap technologies.

### SUMMARY OF THE INVENTION

The subject invention is directed to a therapeutic compression system and methods of use. The therapeutic compression system includes a therapeutic compression apparatus including an air bladder that has a transparent or translucent bladder portion over the needle access site for visualization of any bleeding issues. The therapeutic compression apparatus also includes an inflation means wherein the inflation means is less bulky than known inflation means. The inflation means includes at least two settings of constant inflation or pressure. An optional setting may befor varying or intermittent inflation or pressure. The inventive therapeutic compression system may optionally include at least one sensor, such as a motion sensor, pressure sensor, blood pressure sensor, tannometer sensor, or other sensors to monitor use of the inventive system by the patient and/or medical professionals. The inventive system includes a portion of the compression bladder near to but not limited to the needle access site, such as around the side and back of the arm so as to aid in recovery post-dialysis. The inventive system may also include a relief valve to prevent overinflation as well as a release valve to deflate the inflated bladder.

The system includes different therapeutic compression apparatus such as but not limited to an apparatus for use on a limb such as an arm.

The therapeutic compression system includes a therapeutic compression apparatus which such apparatus may further comprise at least one bladder operatively associated with the therapeutic compression apparatus for applying pressure to a post-dialysis treatment needle access site on the limb such as an arm. The therapeutic compression apparatus includes a connecting means such as a loop and hook material in order to wrap the therapeutic compression apparatus around the limb, such as an arm, of the user. The inventive therapeutic compression apparatus includes a portion of the hook material including an elongated tab configured to connect to an adhesive such as tape allowing the user to place the therapeutic compression apparatus on the arm by himself or herself. Alternatively, the at least one bladder may be integral with the connecting means or wrap. One or more connecting means may be operatively associated along the first and second peripheral edges of the therapeutic compression apparatus for securing it around the limb.

The at least one bladder may be adapted and configured to form a set pressure or compression level when the at least one bladder is filled. Bladder includes a portion which is translucent or transparent for visualization of the needle access site. The translucent or transparent portion of the bladder may be in the shape of a ·'U″, kidney or elongated kidney, oval, circle, rectangle or any other known shapes. The at least one bladder may also include a plurality of fluid chambers. The therapeutic compression apparatus, as part of the inventive system, may further comprise at least one means for adjusting pressure coupled to the at least one bladder for controlling an amount of pressure or compression level supplied to the treatment site.

The subject invention is also directed to a bladder assembly for a compression apparatus for providing pressure to a limb. The bladder assembly comprises at least one bladder having first and second flexible walls secured to one another about a peripheral edge thereof to form an air pocket; and at least one spot weld provided in a predetermined location inward of the peripheral edge connecting the first and second walls to one another to define a plurality of chambers within the bladder.

An inflation means for inflating the bladder such as the air pocket through at least one inflation port may be provided in the first wall of the bladder assembly. The inflation means may be detachable from the at least one inflation port. At least one pressure valve may be operatively associated with the inflation means for controlling an amount of pressure within the bladder and the air pocket within the bladder. The inflation port includes a check value so as to maintain a given pressure within the bladder of the therapeutic compression apparatus. The inflation port may be universal in that it is configured to be capable of connecting to and accepting a plurality of inflation sources and inflation means such as a manual pump, mechanical pump, electrical pump, battery-operated pump, static pump, intermittent pump, pneumatic pump, negative pressure source and other variations. The therapeutic compression apparatus may also include a release valve which when activated by a user releases the air within the air bladder to deflate and release the pressure on the arm of the patient.

The bladder is connected to an inflation port including a valve configured so that when the valve is in the closed position the pressure profile and/or compression profile is maintained at the then current pressure and/or compression level. The valve is configured so that when it is in the open position the fluid, such as air, flows from the inflation means into the bladder and if not connected to an inflation means then the fluid escapes from the bladder and it is deflated. The valve may be self-sealing or it may be connected to a means to open and close it with for example a level, knob, screw or other opening and closing means.

A method of the invention includes the therapeutic compression system including a therapeutic treatment apparatus used to treat swelling and bleeding at a needle access site for the patient after hemodialysis, by applying the primary and/or secondary wraps around a limb by a patient and inserting an inflation means into an inflation port and inflating the bladder within each of the primary wrap and if applicable secondary wrap and maintaining a certain pressure to treat the swelling or bleeding at the needle access site.

Another embodiment of the present invention includes an assembly according to the invention includes a pressure mechanism having a flexible member for attachment to a limb and an air chamber which may be pumped up into a desired pressurized state, a separate relatively small pre-filled air bladder, an absorbent foam, sponge or dressing coupled to the pre-filled air bladder. Optionally a suction conduit may be coupled to a source of negative pressure (suction) and in fluid communication with the absorbent foam, sponge or dressing. In a preferred embodiment, the pre-filled air bladder, the absorbent foam, sponge or dressing and the suction conduit are formed together as a unit. In one embodiment a disk 455 having an indentation or cut-out for the needle lying on the skin of the patient at the needle access site is placed on the skin prior to the therapeutic compression apparatus being placed on the patient wherein the disk may be a foam, gauze or other material. The disk 455 assists in keeping the needle from moving while the inventive therapeutic compression apparatus 100, 200, 300 is placed on the arm 600 and closed via the hook and loop means 122, 222, 322 and 124, 224, 324 and the bladder 102, 202, 302 inflated. At a later time the needle is then removed from under the activated and inflated inventive therapeutic compression apparatus 100, 200, 300. The disk 455 also assists in controlling bleeding once the needle is removed from the patient's arm. The disk 455 could include a gauze portion as well as other medicines to prevent infection at the needle access site.

According to one aspect of the invention, the flexible member of the pressure mechanism is adapted to wrap around an arm and over the pre-filled air bladder in order to secure the pre-filled air bladder and the foam, sponge or dressing to a wound or ulcer in the extremity. Thus, the flexible member is provided with some fixation structure such as a hook and loop closure mechanism. An air pumping mechanism is preferably coupled to the air chamber of the pressure mechanism in order to inflate the air chamber to a pressurized state. The air chamber of the pressure mechanism is preferably designed to apply pressure along a predefined area (e.g., the lower arm or upper arm over the needle access site) as opposed to around an entire limb.

According to another aspect of the invention, the suction conduit is located either between the pre-filled air bladder and the absorbent foam, sponge or dressing which is adhered to the small air bladder, or the pre-filled air bladder is formed as a donut with a central opening and the suction conduit extends through the central opening. By coupling the suction conduit to a source of negative pressure, exudate from the bleeding needle access site is sucked through the foam, sponge or dressing into the suction conduit.

One of the methods of the invention include locating the pre-filled air bladder and foam, sponge or dressing over a bleeding needle access site on an arm, wrapping the flexible member of the pressure mechanism around a limb with the air chamber located over the pre-filled air bladder/absorbent foam (PFAB), sponge or dressing, and fastening the pneumatic pressure mechanism in place with the fixation structure. When the apparatus is properly located and affixed to the lower arm or upper arm of two wraps located on each of the lower arm and upper arm at the same time, the air chamber is inflated, preferably to 30-40 Hg, thereby applying pressure to the limb and more specifically via the pre-filled air bladder to the wound. The suction apparatus is activated by turning on the source of negative pressure, and exudate from the wound or ulcer is pulled through the absorbent foam, sponge or dressing into the suction conduit.

In another embodiment of the present invention, the therapeutic compression system includes an inflation means which has one pressure level such as skin-to-bladder interface pressure of 60 mm-Hg or includes a switch between constant static pressure levels (different pressure levels or compression levels such as 20 mm-Hg, 30 mm-Hg, 40 mm-Hg, 50 mm-Hg, 60 mm-Hg, up to 200 mm-Hg, etc.) and intermittent varying pressure levels, whereby the pressure is applied to the therapeutic compression apparatus via a hose or tube.

In other embodiments the inflation means is connected to two or more tubes and thereby connected to two or more bladders, whether multiple bladders within one therapeutic compression apparatus or a single bladder within multiple therapeutic compression apparatuses, or a combination thereof.

In another embodiment the system includes one or more sensors to monitor movement of the therapeutic compression apparatus, pressure levels, blood pressure of the patient, tonometry of the target limb, or other sensor data.

These and other aspects of the contacts of the subject invention will become more readily apparent from the following description taken in conjunction with the drawings.

### BRIEF DESCRIPTION OF THE DRAWINGS

So that those having ordinaly skill in the art to which the subject invention pertains will more readily understand how to make and use the apparatuses of the subject invention, preferred embodiments thereof will be described in detail herein below with reference to the drawings, wherein:

FIG. 1 is an embodiment of the present invention including a lower arm therapeutic compression apparatus including a transparent portion of the air bladder in the shape of an elongated kidney shape in use on a patient post-hemodialysis treatment including two pieces of gauze on each of the two needle access sites with the needles removed and bladder in an inflated activated state;

FIG. 2 is another view of one embodiment of the present invention including a lower arm therapeutic compression apparatus including a transparent portion of the air bladder in the shape of an elongated kidney showing the outer view which could be used on a left lower arm;

FIG. 3 view of one embodiment of the present invention including a lower arm therapeutic compression apparatus including a transparent portion of the air bladder in the shape of an elongated kidney showing the outer view which could be used on a right lower arm;

FIG. 4 is a further embodiment of an upper therapeutic compression apparatus including a transparent portion of the air bladder in the shape of a kidney for use on the left upper arm or forearm of a user;

FIG. 5 is a further embodiment of an upper therapeutic compression apparatus including a transparent portion of the air bladder in the shape of a kidney for use on the right upper arm or forearm of a user;

FIG. 6 is a further embodiment of an upper therapeutic compression apparatus including a transparent portion of the air bladder in the shape of a circle and a longer length of the bladder and hook and loop closure means approximately 6 inches long for use on either the lower arm or upper arm or forearm of a user and including an extended portion of the tab for self-application with adhesives such as common tape;

FIG. 7 is further embodiment of an arm therapeutic compression apparatus including a transparent portion of the air bladder in the shape of a circle with indentations to form a cross-like shape when inflated and a longer length of the bladder and hook and loop closure means approximately 8 inches long for use on either the lower arm or upper arm or forearm of a user and including an extended portion of the tab for self-application with adhesives such as common tape;

FIG. 8 is a further embodiment of an arm therapeutic compression apparatus including a transparent portion of the air bladder in the shape of a circle and a longer length of the bladder and hook and loop closure means approximately 10 inches long for use on either the lower arm or upper arm or forearm of a user and including an extended portion of the tab for self-application with adhesives such as common tape;

FIG. 9 is a further embodiment of an upper therapeutic compression apparatus including a transparent portion of the air bladder in the shape of a circle and a longer length of the bladder and hook and loop closure means approximately 12 inches long for use on either the lower arm or upper arm or forearm of a user and including an extended portion of the tab for self-application with adhesives such as common tape;

FIG. 10 is a perspective view of FIG. 7 also including a release button;

FIG. 11 is an blown up view of FIG. 7 also including a release button;

FIG. 12 is a back view of a further embodiment of an arm therapeutic compression apparatus including a transparent portion of the air bladder in the shape of a circle and for use on either the lower arm or upper arm or forearm of a user with the bladder in an activated inflated state;

FIG. 13 is an exploded view of FIG. 12 viewed from the front side of the arm therapeutic compression apparatus including an inner sheet and an outer sheet joined together to form the bladder;

FIGS. 14A-C is an embodiment of an inflation means of the present invention with FIG. 14A a front view of the inflation means, FIG. 14B an exploded view of the inflation means of FIG. 14A, and FIG. 14C a blown up view of the vent button assembly;

FIGS. 15A-G are blown up view of portions of the vent button assembly of FIG. 14C which is part of the inflation means of FIGS. 14A and 14B;

FIGS. 16A and 16B are an exploded view of a further embodiment of the inflation means connected to the inflation port on an embodiment of a therapeutic compression apparatus of the instant invention;

FIG. 17 is an embodiment of the present invention (such as FIGS. 6-12) including an arm therapeutic compression apparatus including a transparent portion of the air bladder in the shape of a circle in use on a patient post-hemodialysis treatment including a foam disc having a cut out configured for placement of the needle when the therapeutic compression apparatus is placed and wrapped on the arm two and the bladder in an inflated activated state;

FIG. 18 is an embodiment of the present inventive system including a lower right arm therapeutic compression apparatus including a transparent portion of the air bladder in the shape of an elongated kidney for use on the right lower arm of a user, an inflation means including a hand pump and a gauge, a piece of gauze, and a PFAB;

FIGS. 19A and 19B are further embodiments of a therapeutic compression apparatus including a transparent portion of the air bladder in the shape of a square for use on the upper arm of a user of FIG. 19A and shape of a rectangle for use on the lower arm of a user of FIG. 19B; and

FIG. 20 is a further embodiment of a therapeutic compression apparatus including a transparent portion of the air bladder in the shape of a “U” for use on the upper or lower arm of a user.

### DETAILED DESCRIPTION OF THE PREFERRED EMBODIMENTS

Preferred embodiments of the subject invention are described below with reference to the accompanying drawings, in which like reference numerals represent the same or similar elements. One of ordinary skill in the art would appreciate that while the apparatuses discussed herein relate to compression therapy of the upper arm or lower arm, the scope of the invention is not limited to those exemplary applications and may be sized and shaped for the anatomical portion for which compression therapy is needed such as by way of example post-dialysis treatment at a needle access site once the needle is removed from the patient's skin.

The subject invention provides compression to a patient's limbs, including the extremities, including for example, the arm of a user post-dialysis treatment, in a manner that is simpler, less bulky, more practical, more mobile, and more convenient than current systems. Any limb or body part may be compressed by the instant system including a therapeutic compression apparatus such as for instance a foot, calf, thigh, knee, leg, hip, buttocks, waist, torso, ribs, shoulder, arm, hand, fingers, neck, head or the like depending on the treatment or need for compression at such location.

The subject invention provides system for providing compression and preventing swelling and bleeding of a needle access site after hemodialysis treatment and removal of the two needles. The system is provided in in a manner that allows for consistent measuring of the pressure supplied, as well as safe, comfortable, more practical, more mobile, convenient, effective, and self-application by the patient.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either both of those included limits are also included in the invention. Any such range may be estimated to be lower or higher by at least +of 10%.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a”, “an,” and “the” include plural referents unless the context clearly dictates otherwise. Thus, for example, reference to “a stimulus” would include a plurality of such stimuli and reference to “the signal” would include reference to one or more signals and equivalents thereof known to those skilled in the at1, and so forth. The publications discussed herein are provided solely for their disclosure prior to the filing date of the present application. Nothing herein is to be construed as an admission that the present invention is not entitled to antedate such publication by virtue of prior invention. Further, the dates of publication provided may differ from the actual publication dates which may need to be independently confirmed.

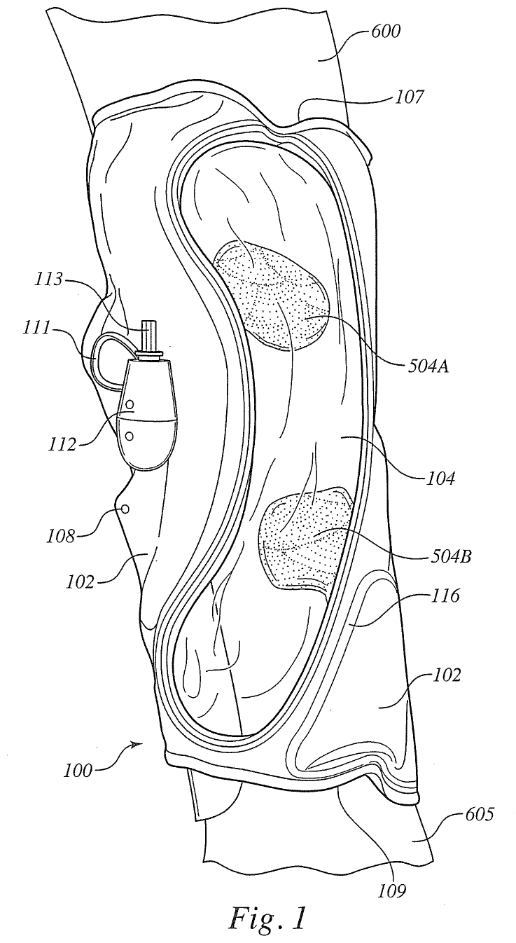

Referring now to FIG. 1, there is illustrated the inventive therapeutic compression system and apparatus in use on the needle access sites on a lower arm of a patient after hemo-dialysis treatment. The inventive therapeutic compression system and apparatus 100 has been placed on the user arm 600, lower arm between the wrist 605 and elbow 610, above the needle access sites over two pieces of absorbent dressing, such as gauze 504A, 504B, and the needles have been removed and the inventive therapeutic compression apparatus 100 inflated and activated. The transparent portion 504 of the bladder shows the location of the needle access sites and the absorbent dressing, gauze 504A and 504B, so that the external bleeding can be monitored at the needle access sites after removal of the needles. This is an example of visualization of lower arm needle access sites which may bleed immediately after the hemo-dialysis treatment or in between treatment sessions. In other patients the needle access sites may be on the upper arm. Also shown is the bladder 102 inflated on the sides of the lower arm adjacent to the needle access sites. The inflation port 112 is closed by the cap 113 and the bladder sealed, typically at interface pressure on the needle access site of 60 mm-Hg though many other pressures may be employed from 10 mm-Hg to over 100 mm-Hg. Either arm may be used and depending on how long this patient has been undergoing hemo-dialysis treatment, there may be multiple fistulas or grafts that stop working and others placed on the other arm or placed on the upper arm after the lower arm locations stop working.

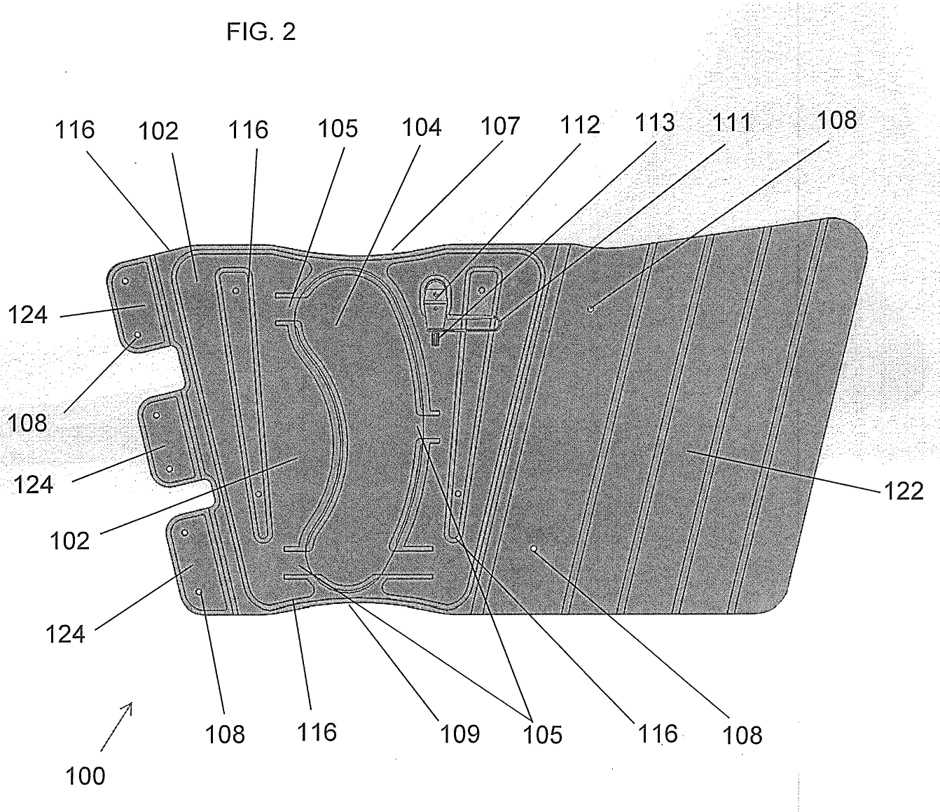

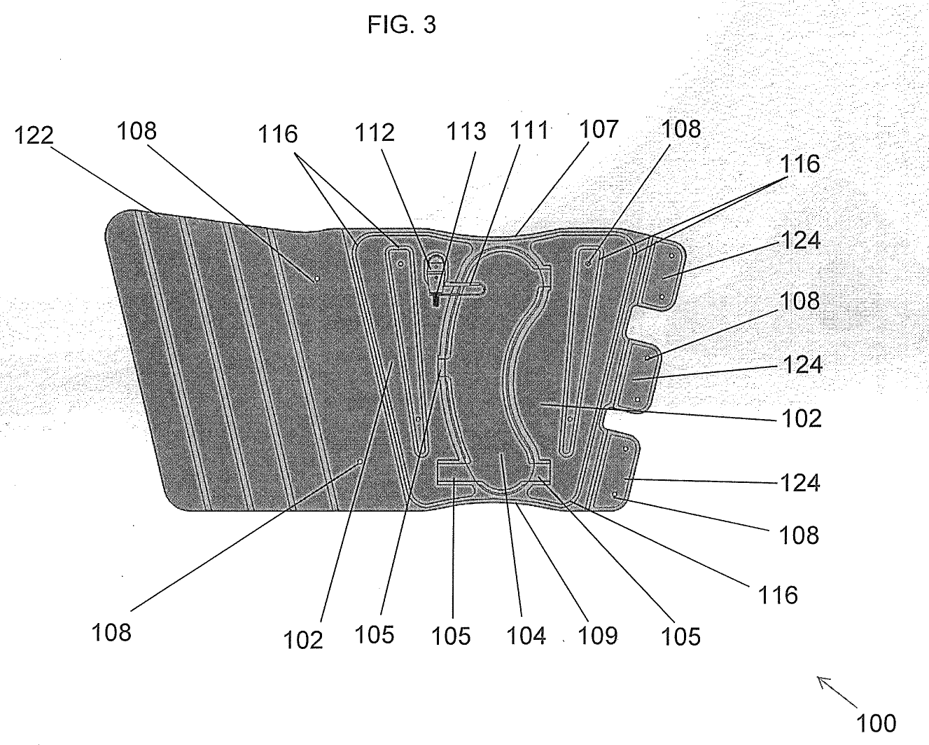

FIGS. 2 and 3 show an exemplary embodiment of the inventive system including a therapeutic compression apparatus 100 for use on the lower arm or forearm of a patient including an elongated kidney shaped transparent portion of the air bladder to allow visual observation of the needle access sites after the hemodialysis treatment. Not shown in these FIGs. are the inflation means 150 and a connecting means 160. In some embodiments the inflation means may be a manual hand pump, foot pump, mechanical pump, electrical pump, battery-operated pump, static pump, intermittent pump, varying pump, automatic pump, pneumatic pump, negative pressure pump, suction pump or vacuum, pulsing pump, or any combination thereof or any other known or developed source of inflation so as to provide a certain pressure within the therapeutic compression apparatus so to provide compression in use by the patient. In some embodiments the inflation means is connected to the therapeutic compression apparatus by a hose or tube. In some embodiments of the inflation means is connected to a retaining means such as a belt or wrap to be worn by the user where an automatic pump is employed for intermittent pressure and inflation followed by deflation.

The therapeutic compression apparatus 100 (and other embodiments shown as 200 and 300) may be comprised of a bladder 102, 202, 302 such as a compression bladder either integrally formed in the therapeutic compression apparatus 100, 200, 300 or the therapeutic compression apparatus 100, 200, 300 is configured for the bladder to be inserted (not shown) within the therapeutic compression apparatus 100, 200, 300. The therapeutic compression apparatus 100, 200, 300 is configured and adapted to wrap around a patient's limb such as in for instance an arm 600 or lower arm or upper arm though other limbs may be included as well such as a leg, calf, knee, foot, ankle, thigh, torso, arm, neck or any other limbs. The therapeutic compression apparatus 100, 200, 300 is not limited to the limbs or body parts listed above but could be any body part such as without limitation a foot, ankle, calf, lower leg, knee, thigh, groin, hip, leg, buttocks, torso, stomach, chest, shoulder, arm, elbow, hand, wrist, neck, head or the like and any combinations thereof. As shown in FIGS. 1-3 the therapeutic compression apparatus 100 is a lower arm apparatus (sometimes referred to as the forearm apparatus) having a transparent portion of the bladder 104 in the shape of an elongated kidney, whereas the therapeutic compression apparatus 200 is an upper arm apparatus as shown in FIGS. 1-3 with the transparent portion of the bladder 204 in the shape of a kidney.

The therapeutic compression apparatus 100 is a wrap member with a proximal end portion 107 (top as oriented in FIGS. 1-3 closer to the elbow 610 of the user) and opposed distal end portion 109 (bottom as oriented in FIGS. 1-3 closer to the wrist 605 of the user) which is configured and adapted to conform around a patient's lower arm and provide compression through the inflation of bladder. The top portion 107 and lower portion 109 of the therapeutic compression apparatus 100 are configured to curve closer to the wrist 605 and/or elbow 610 to fit better or more snuggly on the user when in use.

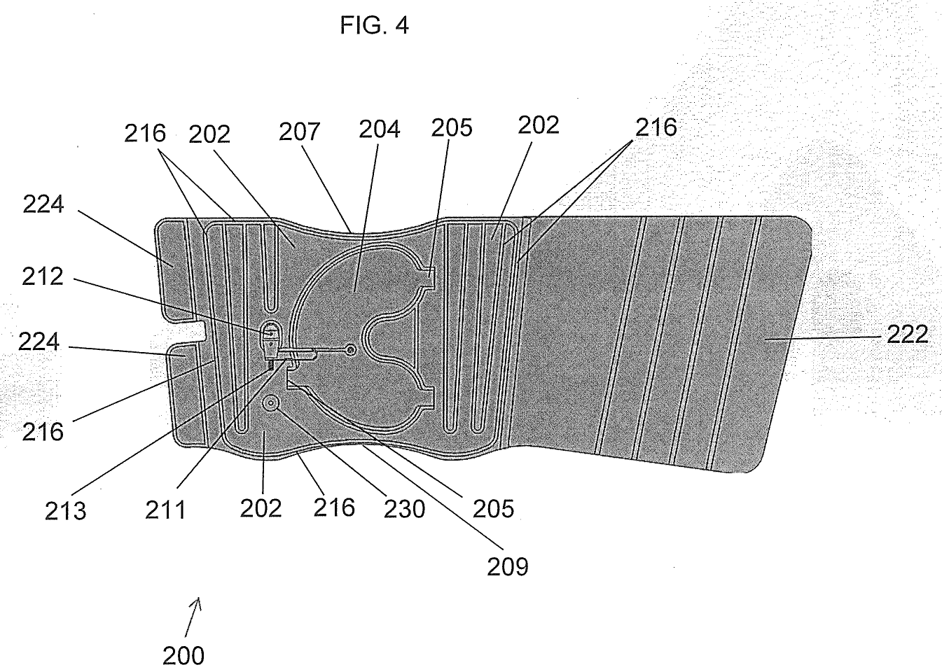

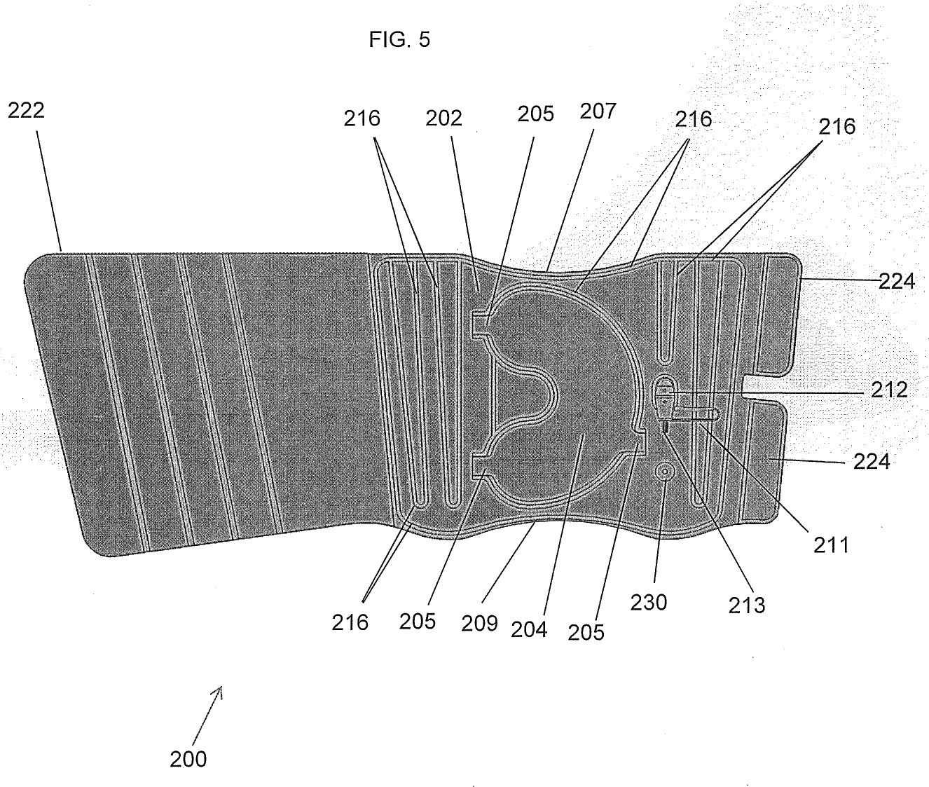

The therapeutic compression apparatus 200 as shown in FIGS. 4-5 is a wrap member with a proximal end potion 207 (top as oriented in FIGS. 4-5 closer to the shoulder 615 of the user) and opposed distal end portion 209 (bottom as oriented in FIGS. 4-5 closer to the elbow 610 of the user) which is configured and adapted to conform around a patient's upper arm and provide compression through the inflation of bladder. The top portion 207 and lower portion 209 of the therapeutic compression apparatus 200 are configured to curve closer to the shoulder 615 and/or elbow 610 to fit better or more snuggly on the user when in use. The top portion 207, 308 of the therapeutic compression apparatus 200, 300 is curved to configure along the elbow 210 if worn on the lower arm or nearer the shoulder 615 if worn on the upper arm. The bottom portion 209, 309 of the therapeutic compression apparatus 200, 300 is curved to configure along the wrist 605 if worn on the upper arm or nearer the elbow 605 if worn on the upper arm. In another embodiment the upper arm apparatus 200, 300 is configured so that it can be turned upside down by the user to make it easier to close the hook and loop portion whether on the right upper arm or left upper arm.

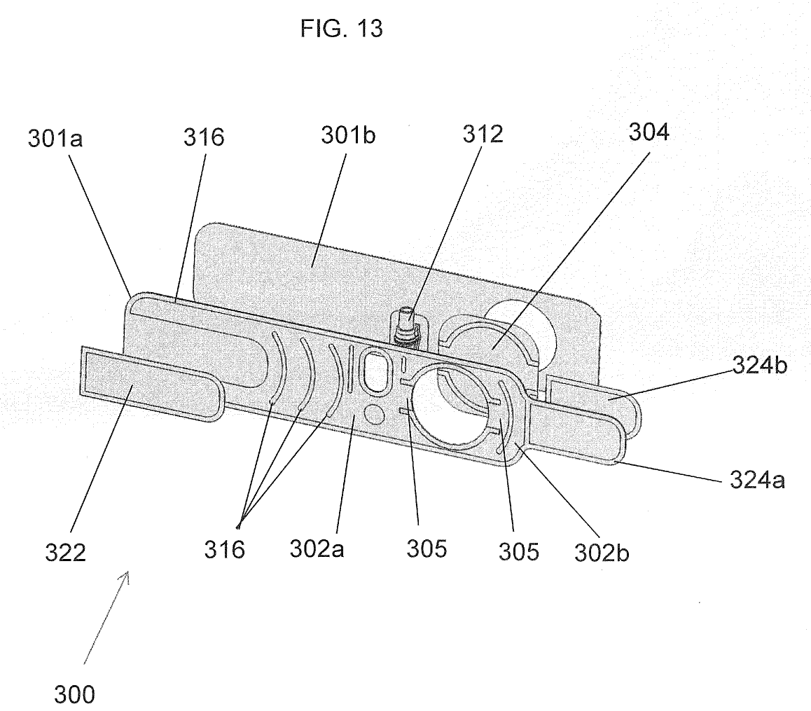

In this embodiment the therapeutic compression apparatus 100, 200, 300 (as shown in exploded detail in FIG. 13) includes an outer sheet 301a and inner sheet 301b which are made out of a nylon laminated polyurethane sheet which are configured and adapted to be RF welded together such as along and via the weld lines 116, 216, 316 which for the boundary of the bladder 102, 202, 302 as well as the compression points and the location of the line welds 116, 216, 316 affect the pressure on the skin both at the needle access site and on the arm adjacent to the needle access site or even around the whole arm area under the wrap when applied and the bladder inflated. However, any other suitable materials which are weldable or otherwise joined while being airtight can be used. Continuous peripheral weld lines 116, 216, 316 form an airtight boundary of integrally formed bladder 102, 202, 302. In this exemplary embodiment, bladder 102, 202, 302 is a single continuous bladder throughout however, it is envisioned that the therapeutic compression apparatus 100, 200, 300 could have an independent bladder either separately inflatable or inflatable through a one-way valve or other desired inflation/deflationconfiguration and even multiple connected or not connected and separate bladders are possible and envisioned in the inventive therapeutic compression apparatus.

In this embodiment, hook and loop fasteners 124, 224, 324 (the hook component) are provided along the edge of inner sheet and outer sheets (as shown in exploded view of FIG. 13) in order to ease adjustment and secure the therapeutic compression apparatus 100, 200, 300 on a patient's limb such as for example an arm and more specifically either a lower arm or an upper arm by connecting to soft material such as doe skin or other stretchable material 122, 222, 322. It is envisioned that the therapeutic compression apparatus 100, 200, 300 can includes hook and loop fasteners 124, 224, 324 connecting to stretchable material 122, 222, 322, but also could be secured to a patient's arm by other means, such as zippered, buttoned, or be cuff shaped by other such suitable means. Further, it is also envisioned that hook and loop closures systems 122, 124, 222, 224, 322, 324 can be replaced by material similar to that of an wrist strap, elbow strap, shoulder strap, arm strap or any other type of strap (including those described below) and be welded/sewn/attached to bladder for improved comfort.

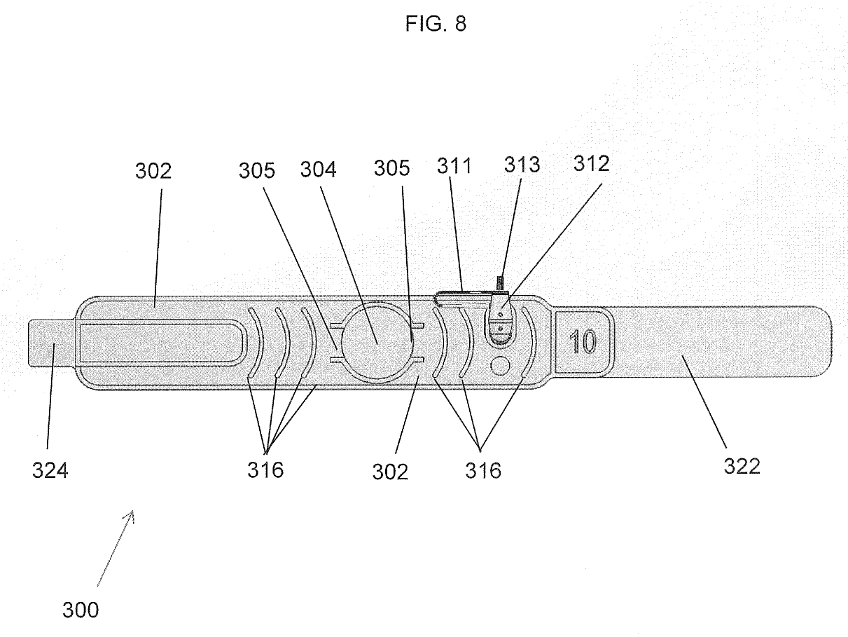

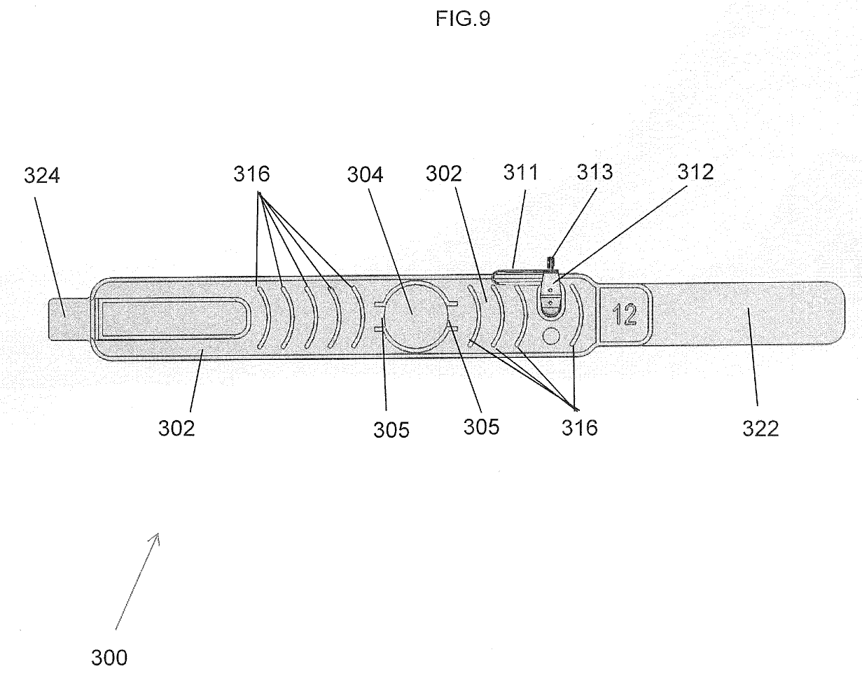

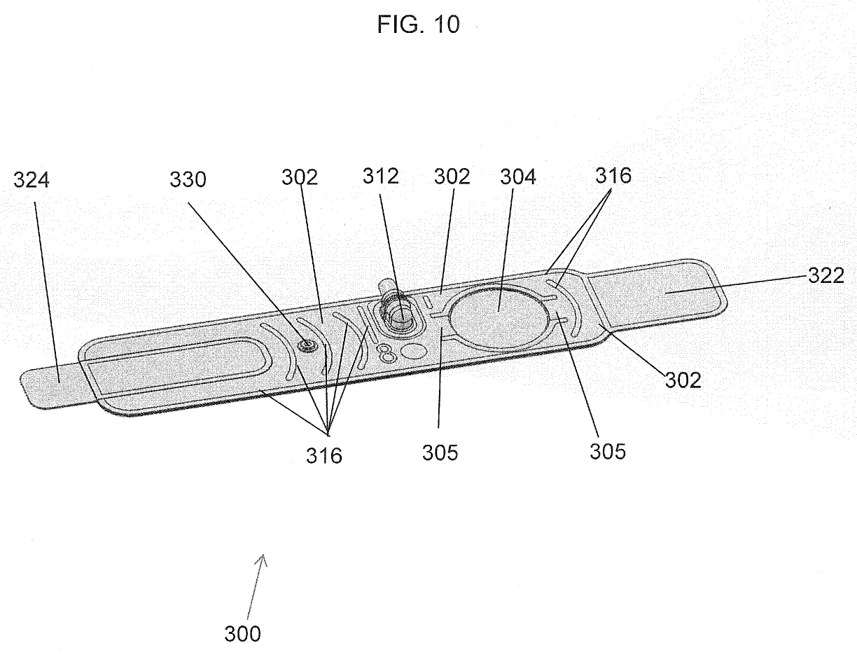

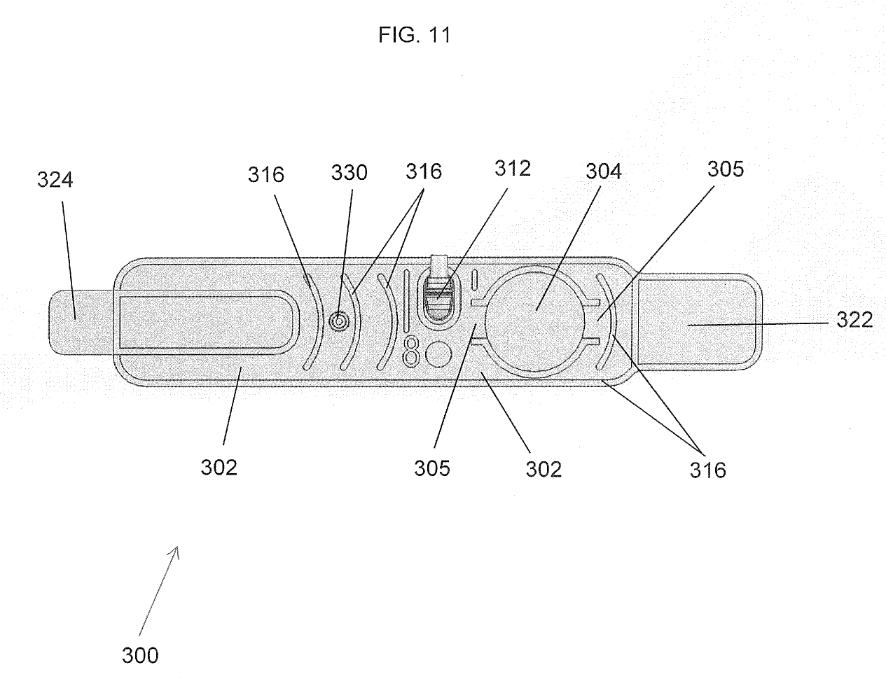

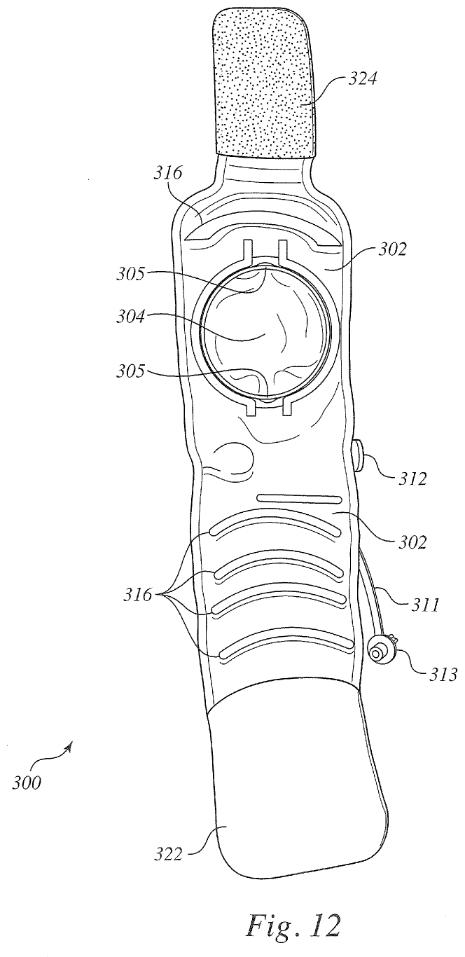

Referring now to FIGS. 2-13 and 18-20, in these embodiments the therapeutic compression apparatus 100, 200, 300 has a bladder integral within outer sheet 101a, 201a, 301a and inner sheet 101b, 201b, 301b, with the location and desired preconfigured compression gradient profile obtained cost-effectively. A number of different embodiments of bladder configurations can be used in the therapeutic compression apparatus 100, 200, 300 such as those configurations described above. FIGS. 2-13 and 18-20 show various inventive therapeutic compression apparatus 100, 200, 300 having bladder 102, 202, 302, with a plurality of line welds 116, 216, 316 therein. Linear weld lines 116, 216, 316 allow for better compression along the arm of a patient. This increased tension can generate a more effective compression in order to reduce or stop external bleeding at the needle access site while also preventing slippage down the arm and securing the transparent bladder portion 104, 204, 304 over the needle access site. The interface pressure has to be sufficient to stop or control bleeding once the needle is removed while still maintaining blood flow within the vessel (vein or artery) under the skin. Linear weld lines 116, 216, 316 located laterally along the arm 600 creating a ribbed portion, which keeps the inflated profile of therapeutic compression apparatus 100, 200, 300 compact which can further increase ambulation and reduce interference with a patient's clothes. There are a number of air holes 108, 208, 308 to allow for breathing of the skin area below the therapeutic compression apparatus 100, 200, 300 such as to reduce sweating and rashes.

It can be appreciated that depending on the location of the therapeutic compression apparatus 100, 200, 300, different pressure levels or compression levels may be utilized. Other possible pressure profile may be imagined based upon the geometric location of the linear welds 116, 216, 316. For example, in the therapeutic compression apparatus 300, depending on the location of the limb to be compressed, such as the upper arm area, the linear welds 216 may be in different geometric configurations than those shown in FIGS. 2-13 for therapeutic compression apparatus 200, 300 for use on the lower or upper arm area. Further, the compression profile and/or pressure profile may be based on the pressure level from the inflation means alone, or combined with the overall shape of the bladder 102, 202, 302 and the overall shape of the translucent or transparent portion of the air bladder which is configured to be placed over the needle access site.

The therapeutic compression apparatus 100, 200, 300 includes a portion of the bladder 100, 202, 302 which is translucent or transparent 04, 204, 304. This translucent or transparent portion 104, 204, 304 of the bladder 2102, 02, 302 is connected to the other portion of the air bladder 102, 202, 302 via openings 105, 205, 305 shown in the FIGURES. The location of the openings 105, 205, 305 may vary in width and height also depending on the shape of transparent portion 104, 204, 304 and the embodiment depending on the application to the lower arm or upper arm when in use.

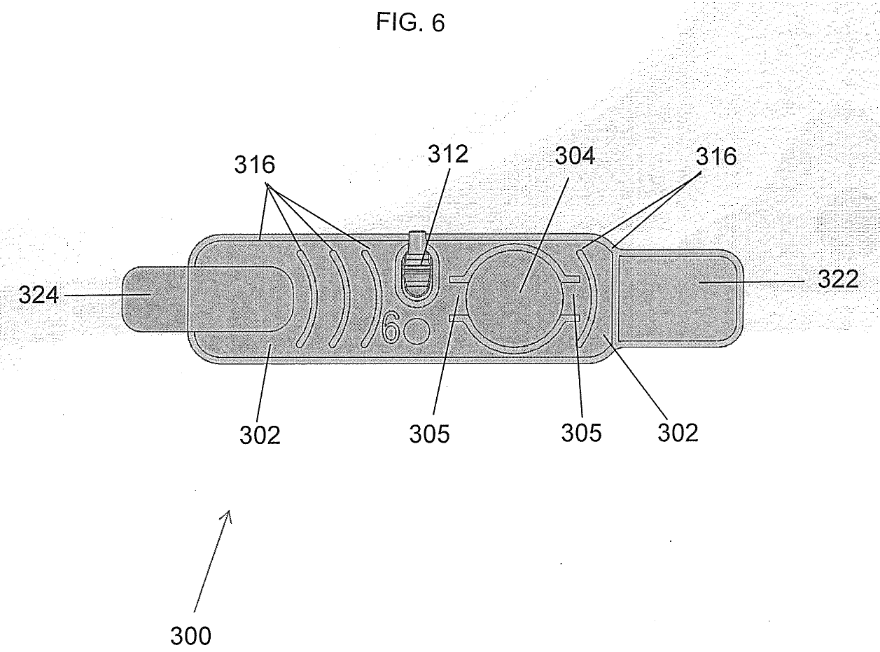

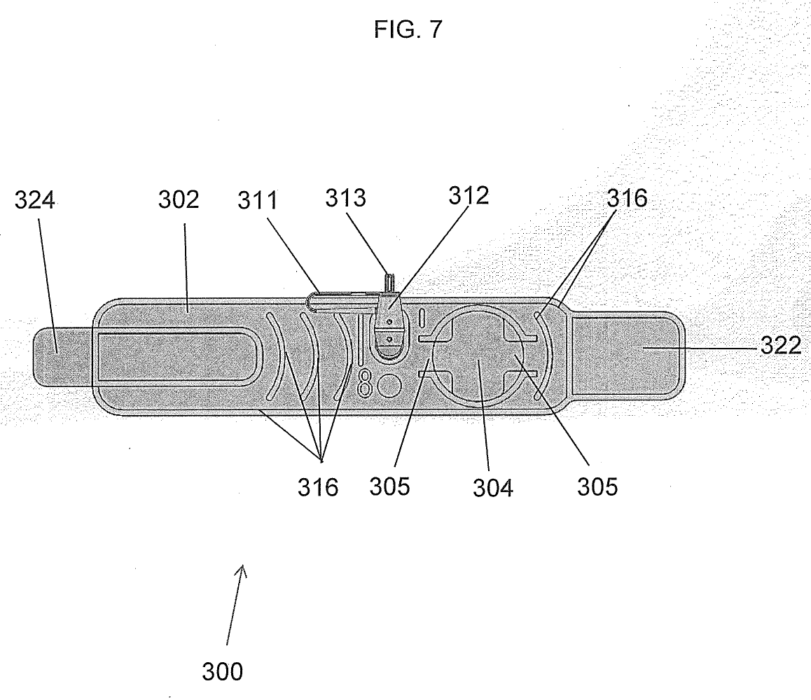

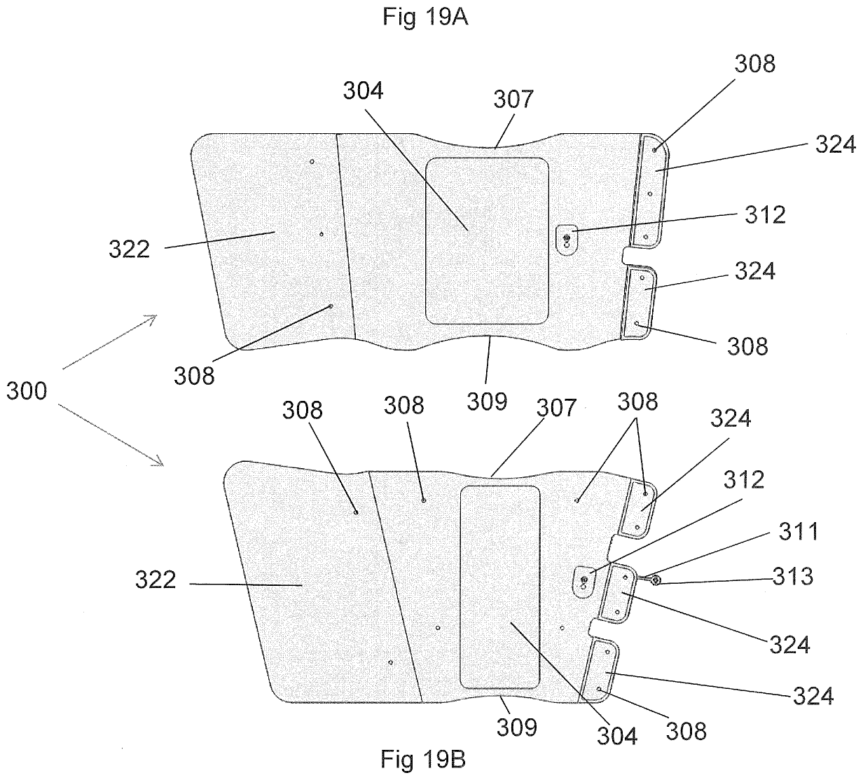

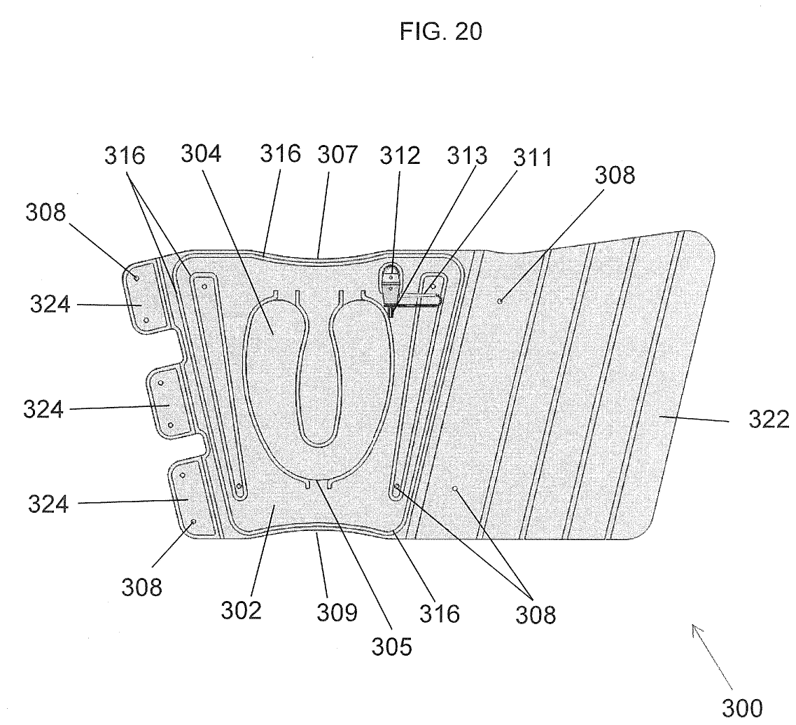

This translucent or transparent portion 104, 204, 304 of the bladder 102, 202, 302 is configured to be placed over the needle access site after hemodialysis treatment so the user or medical staff can visually observe any external bleeding through the gauze or bandage applied over the needle access site once the needle is removed. The translucent or transparent portion 104, 204, 304 of the bladder 102, 202, 302 may be configured in various shapes as shown in the FIGURES including without limitation a rectangle, square, kidney, elongated kidney, “U”, “C”, oval, circle or any other known shape. FIGS. 1-3 and 18 show the translucent or transparent portion 104, 204, 304 of the bladder 102, 202, 302 in the shape of an elongated kidney. FIGS. 4-5 show the translucent or transparent portion 104, 204, 304 of the bladder 102, 202, 302 in the shape of a kidney. In the embodiment shown in FIGS. 6-13 and 17 the therapeutic compression apparatus 200, 300 shows the translucent or transparent portion 204, 304 in the shape of a circle (FIG. 7 has additional indents to form the shape of a cross within the circle) and are configured for both the lower arm and upper arm. FIG. 19A shows the translucent or transparent portion 104, 204, 304 of the bladder 102, 202, 302 in the shape of a square and FIG. 19B in the shape of a rectangle. FIG. 20 shows another embodiment of the therapeutic compression apparatus 350 in this configuration for the lower arm, with the translucent or transparent portion 354 of the bladder 302 in the shape of a ·'U″. Other known shapes may be employed.

The translucent or transparent portion 104, 204, 304 of the bladder 102, 202, 302 of the inventive therapeutic compression apparatus is configured to apply pressure or compression at the needle access site to control external bleeding and reduce or eliminate swelling of the arm area around the needle access site as well as the arm area overall while maintaining sufficient blow flow within the vessel. The pressure or compression is kept constant through the inflation means in this embodiment though intermittent pressure is also envisioned and in other embodiments which may mimic the pulsing of blood through the vessel or other intermittent ranges.

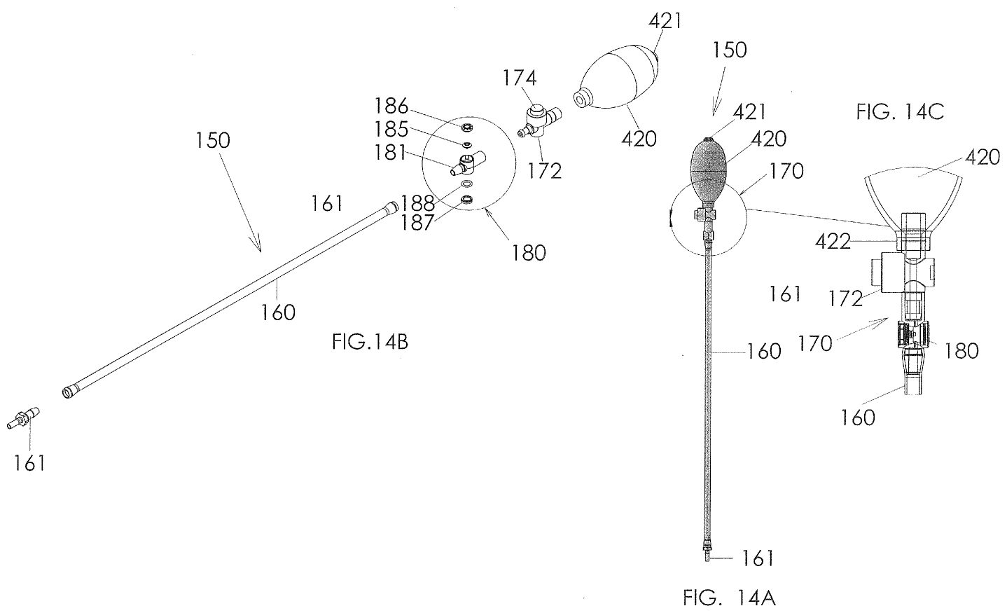

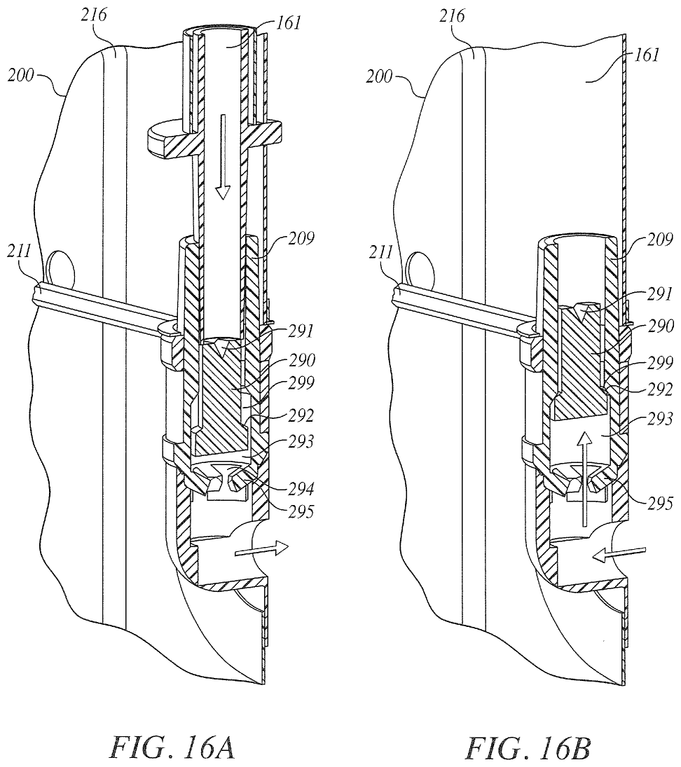

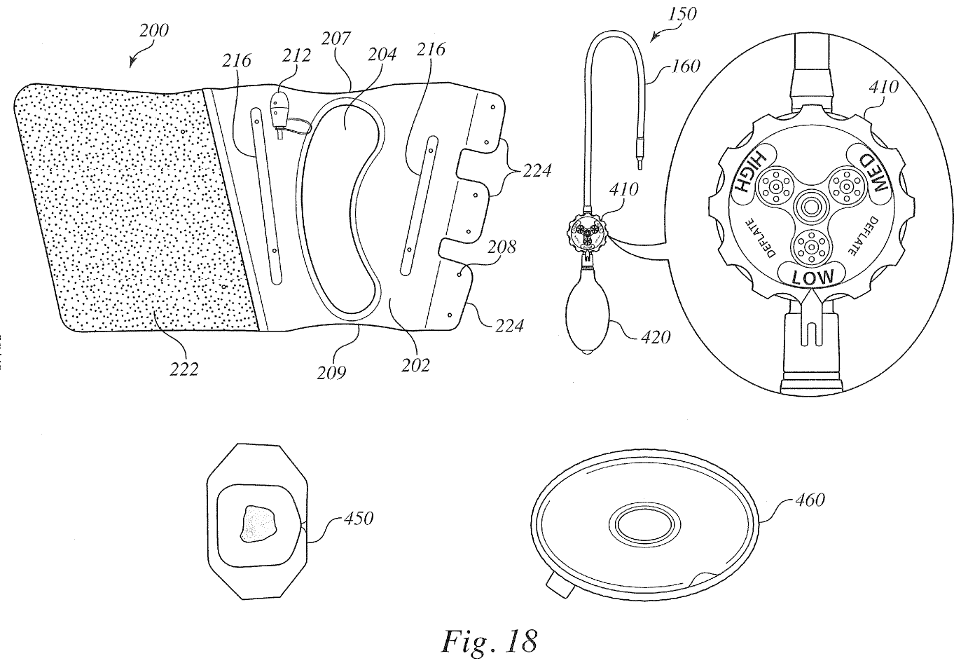

The inflation means may be a hand pump as shown in FIGS. 14-15 and 18. In the embodiment as shown in FIG. 18 the inflation means is a device 150 which is a manual pump capable of attaching to inflation port 112, 212, 312 to inflate bladder within the therapeutic compression apparatus 100, 200, 300 though other pneumatic pumps may be employed. As shown in FIG. 18 the inflation means 150 is a is a hand manual pump 400 including a bulb 420 for manually pumping of air fluid and a hand dial 410 including a check valve within. A number or variety of inflation means can be employed such as a manual pump, hand pump, foot pump, mechanical pump, electrical pump, battery-operated pump, static pump, intermittent pump, varying pump, automatic pump, pneumatic pump, negative pressure pump, suction pump or vacuum, pulsing pump, or any other known or developed source of inflation so as to provide a certain pressure within the bladder so to provide compression in use by the patient. As seen in FIGS. 16A and 16B which is a close up view of a valve 290 incorporated into therapeutic compression apparatus 200 in the inflation port 212 to allow a user to selectively deflate bladder 202 of the therapeutic compression apparatus 200. Further, a check valve (not shown) or relief valve (not shown) is incorporated with either inflation means 150 or bladder 202 to prevent over-inflation once a maximum pressure is detected. Examples of relief valves are described in U.S. Pat. No. 7,276,037 and U.S. Pat. No. 7,850,629, the disclosures of which are incorporated by reference in their entirety. In another embodiment not shown it can be appreciated that other mechanical or automatic inflation pump (not shown) can also be attached to inflation port 212 to inflate and deflate bladder 202 within the therapeutic compression apparatus 200 to provide pulsating pressure to a user's limb.

The inventive therapeutic system includes an inventive sealing means connected to the bladder 102, 202, 302 so as to maintain the constant compression profile and/or pressure profile when the inflation means 150 is either disconnected from the therapeutic compression apparatus 100, 200, 300 or the inflation means stops providing additional inflation or pressure. In the embodiment shown the sealing means is a cap 113, 213, 313 which is inserted onto or into the inflation port 112, 212, 312. The sealing means also includes a valve 290 within the inflation port 112, 212, 312. As shown in FIGS. 16A-B which is a cut-away of the inflation port 212, the valve 290 is located within a housing 214, the housing being tubing or for instance a luer. In this embodiment the housing 214 is a plastic female luer but other materials can be employed such as metal, polymers, or rubbers and other housing means can be employed. The valve 290 as shown in FIGS. 16A-B includes a V-notch 291 in the top proximal portion of the valve 290 though other shapes of notches or other apertures and openings may be employed. The V-notch 291 in this embodiment assists in movement of the fluid from the male luer 161 (fluid flowing from the inflation means 150) into the inflation port 112, 212, 312 and into the bladder 102, 202, 302. The bottom distal portion of the valve 290 is in a shape as shown such as a plug where the top portion 291 is thinner than the bottom distal portion and has a location 292 which abuts and seals the inflation port when it rests along the top portion 299 of the indent 293 in the lower portion of the housing 209. When the two portions 292 and 299 are connected or touching, the inflation port 112, 212, 312 is sealed and the fluid within the therapeutic compression apparatus 100, 200, 300 is sealed within the bladder 102, 202, 302 and thus the pressure and compression within is sealed and if applicable the gradient compression profile and/or gradient pressure profile is maintained. As seen in FIGS. 14A-14B, the housing 214 has a circumference which expands in a lower distal portion to house the valve 290. The shape as shown is not limiting and any other shapes may be employed, as long as some portion of the valve 290 touches a portion of the housing 214 so as to create a sealing means and maintain the pressure and fluid within the inflated therapeutic compression apparatus 100, 200, 300. Again other shapes may be employed such that the functionality is created for a sealing means. The bottom distal portion 295 of the housing 214 in this embodiment then narrows or tapers so that when the valve 290 is pushed down by the male luer 161 the bottom of the valve 290 rests on top of 295 and the fluid passes around the outer circumference of the valve 290 and also through the V-notch 291 and flows into the bladder 102, 202, 302 via the opening or aperture 294. The outer circumference of the bottom distal portion of the valve 290 is slightly smaller than the inner circumference of the bottom distal portion 293 of the housing 214 so that the fluid can flow from the inflation means 150 through the hose 160 through the slip luer 161 through the inflation port 112, 212, 312 and into the bladder 102, 202, 302.

FIG. 16A shows the inflation of the therapeutic compression apparatus 200 with the male slip luer 161 inserted within the housing 214 of the inflation port 112, 212, 312 (the optional cap 113, 213, 313 is open and off to the side) and pushes down on the valve 290 so that the valve 290 bottom rests on the bottom distal portion 295 of the housing 214 which is referred to as the open position for the valve 290. The fluid represented by the downward arrow in the middle of the male slip luer 161 flows into the housing 214 and past the valve 290 and into the bladder 202 as shown by the arrow direction in the bottom of the figure. The fluid will continue to flow into the bladder 102, 202, 302 until such time as the therapeutic compression apparatus 100, 200, 300 is in the appropriate inflated state. The user then removes the maleslip luer 161 inserted from the housing 214 of the inflation port 212 (as shown in FIG. 16B) and the valve 290 moves in an upward direction so that the valve 290 bottom distal portion 292 touches the corresponding shape of the top portion 299 of the indent 293 in the lower portion of the housing 214. The two portions 292 and 299 are connected or touching thereby sealing the inflation port 212 and the fluid within the therapeutic compression apparatus 100, 200, 300 is maintained at the level prior to the inflation means 150 being disconnected to therapeutic compression apparatus 100, 200, 300. The fluid is represented by the upward arrow in the middle of the housing 214 flowing from the bladder 202 and into the bottom portion of the indented housing 293 is sealed by the valve 290. The inflation means 150 could be a manual pump 400 as well and any other static or intermittent inflation means. When the bladder 102, 202, 302 is deflated by the user the valve 290 will again be pushed down by the male slip luer 161 but no fluid or inflation will be employed at that time so that the fluid flows out of the inflation port past the valve 290 and the deflated state will be maintained. The valve 290 can be manually pushed down to deflate by inserting the male slip luer 161 or by a stem or other extending portion of the cap 113, 213, 313 or by any other appliance to push the valve 290 in a downward direction. In this embodiment the valve 290 is self-sealing but other sealing means can be employed such as any moveable lever, screw, switch, stop cock, or other mechanical means to seal the inflation port and maintain the fluid within.

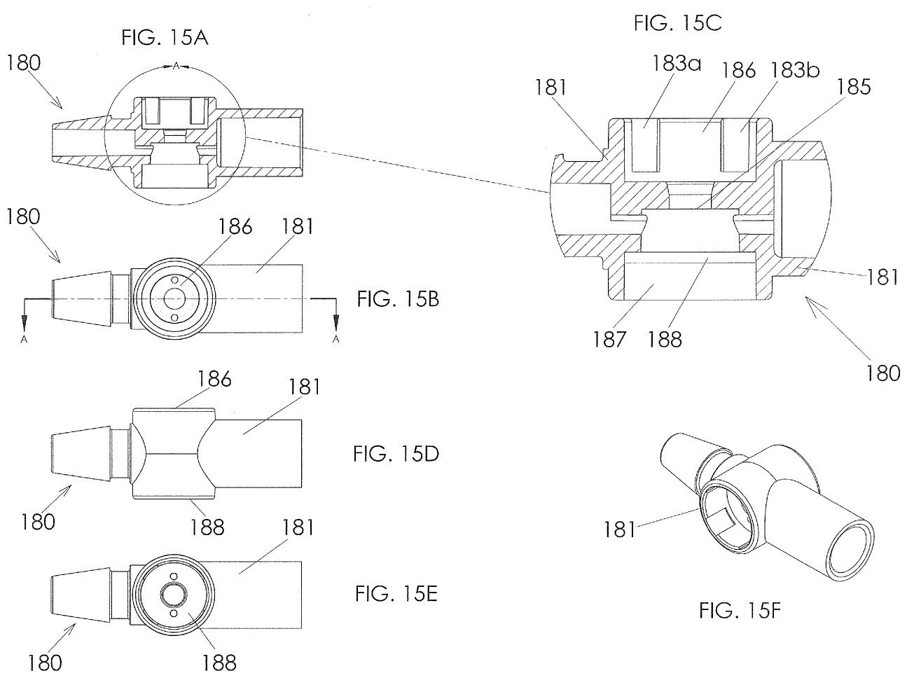

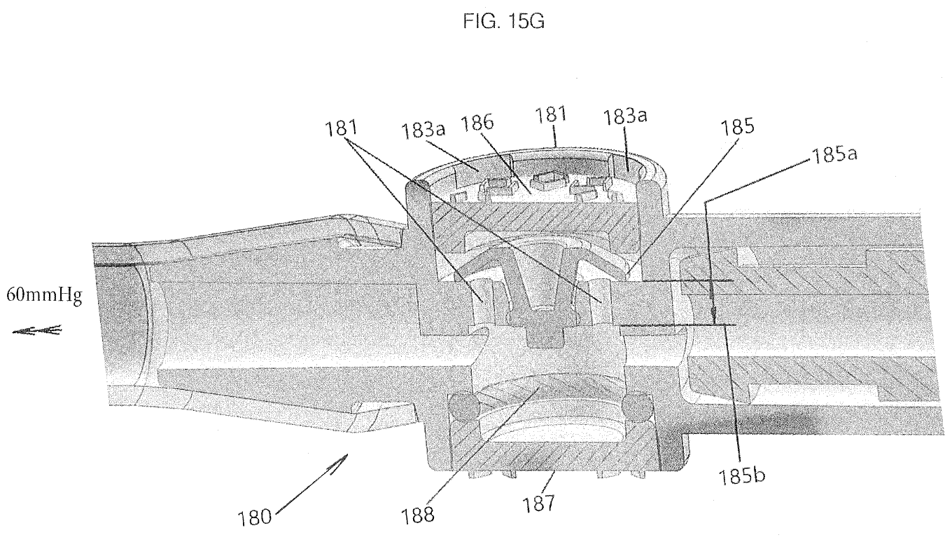

FIGS. 14A-C and FIGS. 15A-G show an inventive vent button assembly connected to the hand bulb 420 tube 160 or hose. FIG. 14A is the inflation means 150 of this embodiment of the present invention including the hand bulb 420 including at least one check valve 421 and preferably two check valves 422, a vent button assembly 170, tube 160 or hose, and male luer slip 161. The vent button assembly 170 includes a vent 172 and a pressure relief valve assembly 180 which in this embodiment is configured to target the maximum bladder pressure at a range of approximately 80-140 mm-Hg which corresponds to an interface pressure on the patient at the transparent bladder portion 104, 204, 304 when placed over the needle access site in a range of about 50-70 mm-Hg, preferably about 55-65 mm-Hg interface pressure. The vent 172 assembly also includes a button 172 which when depressed releases the pressure within and deflates the bladder 102 202, 302. The bulb 420 contains a check valve at each end 421, 422, so that when the bulb 420 is squeezed air is forced out of the front of the bulb 420. When the bulb 420 is released, air is pulled into the bulb 420 through the rear check valve 421. When the hand bulb 420 is connected to a pressurized bladder 102, 202, 302, the 170 vent button assembly will allow air flow out of the bladder 102, 202, 302 when the button 174 is depressed.

FIGS. 14B and 15A-G show in more detail the inventive pressure relief valve assembly 180 configured to target the bladder 102, 202, 302 interface pressure at about 55-65 mm-Hg. The inventive pressure relief valve assembly 180 includes an umbrella valve 185 to regulate the maximum air pressure that can be delivered from the bulb 420 to the bladder 102, 202, 302. At low pressure the umbrella valve 185 remains closed and air flow is directed straight through the bulb 420 into and through the valve assembly 180, through the valve housing 181 and into the bladder 102, 202, 302. Once the pre-determined maximum pressure is reached, such as by way of example only an interface pressure 55-65mm-Hg (or any other determined pressure in the range of 40-80 mm-Hg interface pressure), the umbrella valve 185 opens and air from any further additional bulb 410 cycles (more hand pumps of the bulb 420) is directed out of the valve housing 181 via at least one vent slot 183 (183a, 183b as shown in FIG. 15G) and not into the bladder 102, 202, 302. Air flows out of the valve housing 181 through at least one and preferably multiple vent slots (183a, 183b, around the outer diameter of the top plug 186). The top plug 186 is slightly recessed into the valve housing 181 so that a silo is created. The bottom plug 187 is sealed with an O-ring 188 to prevent air leakage out of the valve housing 181 on the bottom side of the vent assembly 180. The umbrella valve 185 is chosen for height (185a, 185b) and durometer of material, such as silicone or other polymers or plastics, based on the pre-determined interface pressure selected as when this blow off pressure is exceeded the umbrella valve 185 flexes and directs excess pressure out of the vent slot 183 (or slots 183a, 183b, 183c, 183d, etc.) and therefore the pre-determined maximum interface pressure for the needle access site is not exceeded which if exceeded could cause complications based on impediment of blood flow through the vessels under the skin. As shown in FIG. 15G, the measurement of the heigh of the umbrella valve (185a, 185b) is calculated and set dependent on the pre-determined interface pressure selected for such therapeutic compression apparatus based on the target compression needed for the body part. This inventive safety feature of the inventive therapeutic compression system decreases complications following dialysis treatment at the needle access site and surrounding the needle access site which complications from compression include cutting off blood circulation, compartment syndrome, thrombosis, aneurism or even necrosis.

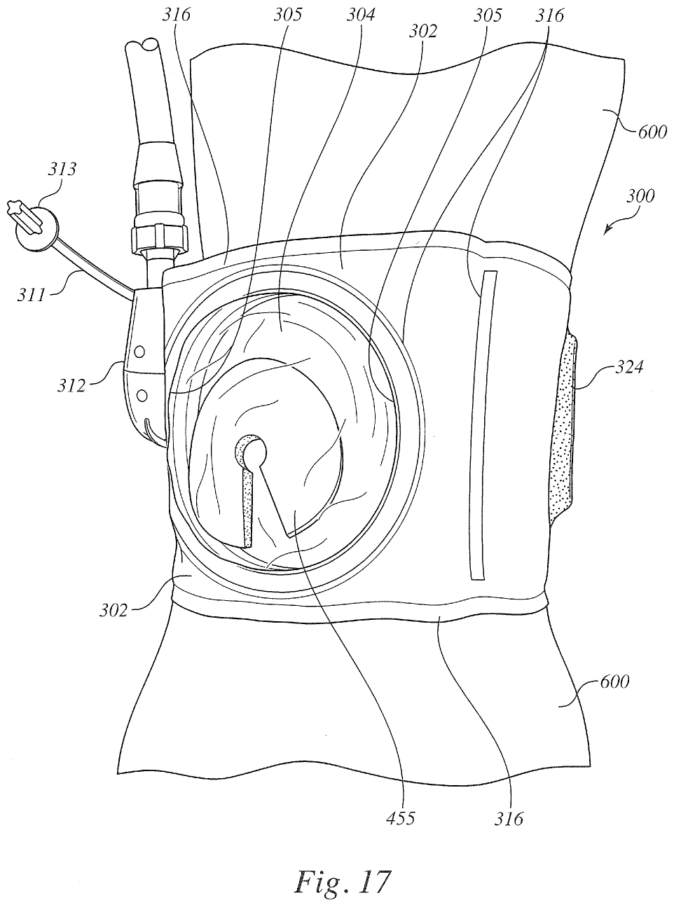

As shown in FIGS. 1 and 17, once the therapeutic compression apparatus 100, 200, 300 is secured around a patient's arm, bladder 102, 202, 302 is inflated and not able to shift out of place via the compression of the inflated bladder 102, 202, 302, thus increasing comfort and reducing fitting issues on the patient. It will be appreciated that the chambers of the bladder 102, 202, 302 may be filled by air, fluid or other known means of inflation. It will also be appreciated that the bladder 102, 202, 302 can be arranged to receive air and be inflated using a manual pumping bulb or can be inflated by an electric air pump (not shown) which can use batteries or AC wall current to pump air into the chamber(s). The inflation port 112, 212, 312 of the therapeutic compression apparatus 100, 200, 300 is universal in that it can connect to a multiple of different types of inflation means. Any known source of air or fluid may be employed whether manual, mechanical, electrical, battery-operated or any other power sourced pump or pressure creator. The inflation means 150 may be a manual pump, hand pump, foot pump, mechanical pump, electrical pump, battery-operated pump, static pump, intermittent pump, varying pump, automatic pump, pneumatic pump, negative pressure pump, suction pump or vacuum, pulsing pump, or any other known or developed source of inflation so as to provide a celtain pressure within the bladder so to provide compression in use by the patient. The inflation means 150 is connected to the therapeutic compression apparatus 100, 200, 300 via a tube 160 or hose. Any other known connecting means may be employed. The embodiments shown in FIGS. 14 and 18 include a tube 160 connected to the handheld manual pump 400. The tube 160 is connected to the therapeutic compression apparatus 100, 200, 300, by inserting, for instance a male luer slip 161, into the inflation port 112, 212, 312. The inventive therapeutic system includes an inventive sealing means connected to the bladder 102, 202, 302 so as to maintain the constant compression profile and/or pressure profile when the inflation means 150 is either disconnected from the therapeutic compression apparatus 100, 200, 300 or the inflation means stops providing additional inflation or pressure. In the embodiment shown the sealing means is a cap 113, 213, 313 which is inserted onto or into the inflation port 112, 212, 312. The sealing means also includes a valve 290 within the inflation port 112, 212, 312. As shown in FIGS. 16A-B which is a cut-away of the inflation port 212, the valve 290 is located within a housing 214, the housing being tubing or for instance a luer. In this embodiment the housing 214 is a plastic female luer but other materials can be employed such as metal, polymers, or rubbers and other housing means can be employed. The valve 290 as shown in FIGS. 14A and 14B includes a V-notch 291 in the top proximal portion of the valve 290 though other shapes of notches or other apertures and openings may be employed. The V-notch 291 in this embodiment assists in movement of the fluid from the male luer 161 (fluid flowing from the inflation means 150) into the inflation port 112, 212, 312 and into the bladder 102, 202, 302. The bottom distal portion of the valve 290 is in a shape as shown in FIGS. 16A-16B such as a plug where the top portion 291 is thinner than the bottom distal portion and has a location 292 which abuts and seals the inflation port when it rests along the top portion 299 of the indent 293 in the lower portion of the housing 209. When the two portions 292 and 299 are connected or touching, the inflation port 112, 212, 312 is sealed and the fluid within the therapeutic compression apparatus 100, 200, 300 is sealed within the bladder 102, 202, 302 and thus the pressure and compression within is sealed and if applicable the compression profile, gradient compression profile and/or gradient pressure profile is maintained. As seen in FIGS. 16A-164B, the housing 214 has a circumference which expands in a lower distal portion to house the valve 290. The shape as shown in FIGS. 164A-16B is not limiting and any other shapes ay be employed, as long as some portion of the valve 290 touches a portion of the housing 214 so as to create a sealing means and maintain the pressure and fluid within the inflated therapeutic compression apparatus 100, 200, 300. Again other shapes may be employed such that the functionality is created for a sealing means. The bottom distal portion 295 of the housing 214 in this embodiment then narrows or tapers so that when the valve 290 is pushed down by the male luer 161 the bottom of the valve 290 rests on top of 295 and the fluid passes around the outer circumference of the valve 290 and also through the V-notch 291 and flows into the bladder 202 via the opening or aperture 294. The outer circumference of the bottom distal portion of the valve 290 is slightly smaller than the inner circumference of the bottom distal portion 293 of the housing 209 so that the fluid can flow from the inflation means 150 through the hose 160 through the slip luer 161 through the inflation port 112, 212, 312 and into the bladder 102, 202, 302.

The inflation means 150 may apply pressure constantly in a static status or may apply pressure intermittently, for example by applying 20 mm-Hg, 25 mm-Hg, 30 mm-Hg, 35 mm-Hg, 40 mm-Hg, 45 mm-Hg, 50 mm-Hg, 55 mm-Hg, 60 mm-Hg, 65 mm-Hg, 70 mm-Hg, 75 mm-Hg, 80 mm-Hg, 90 mm-Hg, 60 mm-Hg, 75 mm-Hg, 80 mm-Hg, 90 mm-Hg, 100 mm-Hg or other known interface pressure levels, depending on the treatment plan and the target limb of the patient. There could also be multiple pressure levels within the pump so there is varying pressure during treatment. In one embodiment of the present invention the interfacial pressure on the needle access site post-dialysis may be between 40-60 mm Hg, preferably about 60 mm-Hg. The interfacial pressure on the needle access site must be sufficient to stop external bleeding once the needle is removed post-dialysis treatment while maintaining blood flow within the vessel so as to prevent complications such as circulation cut off, swelling, edema, bruising, compartment syndrome, thrombosis, aneurism or even necrosis or loss of limb.

The inventive therapeutic compression apparatus 100, 200, 300, may be comprised of both non-elastic and elastic materials, semi-rigid, laminates, polymers, doeskin, stretchable, or other materials sufficient to provide the interface compression of about 20-80 mm-Hg. The inner layer is non-elastic and is preferably made of a wicking, non-hook and loop compatible material, so as to pull perspiration and other unwanted moisture away from the arm area around the needle access site being treated. The non-hooking material against the patient's skin also reduces irritation, which may be caused from a hooking material. The outer layer has at least a portion which is hook compatible in order for the connecting tabs 122, 124, 222, 224, 322, 324 to be secured at the peripheral edge of the inventive therapeutic compression apparatus around the arm, shoulder, elbow, wrist or other limb. For example, the flexible therapeutic compression apparatus 100, 200, 300, may be comprised of two layers of elastomeric material with the air bladder 102, 202, 302 affixed between these two layers by nylon threads or other suitable fastening means. The dimensions of the inventive therapeutic compression apparatus 100, 200, 300 are configured to provide coverage and compression on one or more needle access sites and may be in the range of about 2 inches to about 18 inches or longer in width to wrap around the circumference of an arm, shoulder, elbow or wrist or other body part and a length of about inch to about 18 inches to correspond to the length of a wrist, lower arm, forearm, upper arm, bicep, elbow or shoulder or other body part. The depth is in a range of about 1 mm to about 100 mm depending on the inflation and pressure and compression profile of the inventive therapeutic compression apparatus 100, 200, 300 and the body part being compressed over the needle access site. Other dimensions of length, width and depth may be employed. The translucent or transparent portion 104, 204, 304 of the bladder 102, 202, 302 may have circular radius of about 1 mm to about 500 mm and in other shapes have a length of about 2 mm to about 1000 mm and a width of about 2 mm to about 1000 mm and depth (when inflated) of about 1 mm to about 500 mm. Other dimension of length, width and depth may be employed for the translucent or transparent portion 104, 204, 304 of the bladder 102, 202, 302.

The patient if completing dialysis at home can apply and secure and inflate the inventive therapeutic compression system and apparatus on his or her own. The patient places an absorbent dressing on the needle access site over the needle still within the arm. The absorbent dressing may be gauze, bandages, foam (such as a disk shown in FIG. 17), PFAB or other materials. The patient may put an adhesive such as tape on the extended tab 124, 224, 324 (as shown in FIGS. 6-11) and tapes the extended tab 124, 224, 324 to his or her arm. The patient then wraps the therapeutic compression apparatus 100, 200, 300 around the arm making sure the transparent bladder portion 104, 204, 304 is over the needle access site(s) to allow visualization and monitor for external bleeding. Next the patient can remove the tape from his or her arm. The patient then inflates the bladder 102, 202, 302 via the inflation means 150, such as by inserting the luer 161 within the inflation port 112, 212, 312 and squeezing the hand bulb 420 repeatedly until such time as the bladder 102, 202, 302 is fully inflated and compression is felt on the needle access site and on the arm adjacent to the site or even on the entire circumference of the arm at that area, or shoulder, elbow or wrist are of the inventive therapeutic compression apparatus is located on the shoulder, elbow or wrist part of the arm. The patient or user activates the manual hand pump 420 and stops at the decided pressure level and removes the male luer slip 161 and depending on which embodiment of the inventive therapeutic compression apparatus is employed there is either a self-sealing valve or the patient inserts the cap 113, 213, 313 into the inflation port 112, 212, 312. Finally, the patient or a medical provider removes the needle from below the inventive therapeutic compression apparatus 100, 200, 300. Once the therapeutic compression apparatus 100, 200, 300 is secured around a patient's limb such as for instance an arm 600, the inflated bladder 102, 202, 302 is not able to shift out of place, thus increasing comfort and reducing fitting issues on the patient. The user thus has increased mobility compared to a therapeutic compression apparatus where the inflation means is integral to the apparatus. The compression may be applied to the needle access site for a period of time to stop externally bleeding which is dependent on the patient (thin blood, clotting parameters, and other variants) and can be from a period of about 15 minutes to hours. The inventive therapeutic compression apparatus may be adjusted via the hook and loop securing means 122, 124, 222, 224, 322, 324 if the compression feels too tight to the patient or user or too loose, as well as the pressure adjusted to reduce or increase the interface pressure level.

The inflation means could include a means to monitor or regulate the inflation. The inflation means could include programming such that the bladder 102, 202, 302 is inflated and deflated to a set pressure at intervals or at set times throughout the day or night when the compression apparatus is in use worn on the patient. For instance, by way of example only, the inflation means could be set to 60 mm-Hg at 9 am and then set to deflate to 40 mm-Hg at 11 am and then set to inflate to 40 mm-Hg at 12 pm and so on throughout the day and night for each patient individually. In another embodiment of the present invention, if the therapeutic compression apparatus has two separate bladders (not shown but for instance bladders 202a and 202b) then there may be two separate inflation ports 212 (e.g. 212a and 212b not shown) that are each connected to either the same or different inflation means, and the pressure levels of the first bladder 202a and the second bladder 202b could be the same or have different pressure levels. For instance, by way of example only, the inflation means could be set to 40 mm-Hg for the first bladder 202b and set to 60 mm-Hg for the second bladder 202b, or each could vary and the inflation means be set to inflate, deflate, inflate, etc. throughout the day as described above.

In one embodiment of the invention, the inflation means includes a manual pump (as shown in FIG. 18) and the dial includes graphics of pressure amount such as “35, “45′, “55′ and “65” or lettering such as “A”, “B”, “C”, “D” which each would correspond to a certain pressure such as 35 mm-Hg, 45 mm-Hg, 55 mm-Hg and 65 mm-Hg. The specific pre-determined pressure to correspond with the graphic is endless and not limited by the examples herein.

Further, the therapeutic compression apparatus 100, 200, 300 may be deflated by a button or a switch 130, 230, 330 to deflate the bladder 102, 202, 302 and thus release the pressure and/or compression profile as shown in FIGS. 4-5 and 10-11. In another embodiment (not shown), the switch may have a plurality of integrated umbrella valves so that one umbrella valve is set and closed to maintain the pressure within the bladder 102, 202, 302 while a second umbrella valve would release a certain amount of air or fluid within the bladder 102, 202, 302 so as to release the pressure such as while the patient is walking (the arm 600 is straight and pressure or blood and bleeding may increase in the wrist 605 as the person walks) or flying (pressure increases based on altitude), and a third umbrella valve which would release all the air or fluid in the bladder 102, 202, 302 thus release all pressure and deflate the therapeutic compression apparatus 100, 200, 300). For instance by way of example only, the first umbrella valve is set in a closed position so that when activated this umbrella valve maintains the amount of air or fluid in the bladder 102, 202, 302 and thus maintains the set pressure, say for instance at 60 mm-Hg, the second umbrella valve is set to release the air or fluid within the bladder 102, 202, 302 if the pressure within exceeds 65 mm-Hg and bring the pressure down to 60 mm-Hg (such as when in high altitude or other increases in pressure when the arm is down while walking) and then maintain the pressure at 60 mm-Hg, and a third umbrella valve is set to open and release all the air or fluid within the bladder 102, 202, 302 and thus release all pressure when activated by the patient so as to deflate the bladder 102, 202, 302 and the therapeutic compression apparatus 100, 200, 300. In all of the embodiments referring to umbrella valve the umbrella valve may also be a switch (manual or otherwise) or a digital switch or any other known means to open, close or partial release air or fluid within a bladder and thereby maintain, change or release pressure therein.

The inventive system may be included in a kit including a therapeutic compression apparatus 100, 200, 300, an inflation means 150 and a connecting means such as a tube 160. The kit may also include gauze 500A-B, 450 and bandages, or PFAB 460 or a foam disk 455 for the user to apply over the needle access site immediately after the hemodialysis treatment or in between treatments sessions if bleeding occurs. An exemplary embodiment of the system or kit is shown in FIG. 18. The kit may also include various wound dressings and/or bandages (500A-B and 450) which may include adhesive for placement on the skin of the user. The PFAB 460, wound dressings and/or bandages may be disposed of on a more frequent basis and the inventive therapeutic compression apparatus is applied in conjunction or combination with the wound dressings and/or bandages. In one embodiment the therapeutic compression apparatus is used over or on top of the wound dressing applied to the skin.

The system may also include other sensor such as a tonometer, which is a device pressed into the skin to measure the amount of force required to make an indent in the tissue. The resulting measurement can help gauge the degree of firmness or fibrosis (tissue scarring) under the skin, which is a consequence of loss of blood circulation. Such a tonometer could be applied to the patient's skin under the therapeutic compression apparatus 100, 200, 300 and measure the firmness or fibrosis at such treatment site on the limb of the patient. Such a tonometer could be connected to the inflation means via Bluetooth or other digital means and provide feedback to the patient and medical staff as discussed above. A pulsometer could also be employed to measure the pulse near the needle access site to ensure sufficient and adequate blood flow for the vessel is maintained, while still stopping the external bleedings at the needle access site. Other sensors may be employed depending on the needs of the patient and user.

In another embodiment, the inventive therapeutic compression system has sensors that measure the positioning and movement of the therapeutic compression apparatus 100, 200, 300. Such sensors could be interpreted to give readouts (via the interface, a plug-in to a computer, or sent to an app) to the user or clinician as to the compliance, activity (pedometer to measure steps, stairs climbed, etc.), and give advice/alerts to improve the treatment. In one embodiment the sensors would be able to measure skin fibrosis and adjust the level of compression required to improve swelling reduction or a pulsometer to release compression and swelling if the vessel is being cut off and circulation impeded. These sensors listed are not limiting and any other known or future developed sensors could be employed with the inventive system.

The sensors described above are non-limiting and could be non-digital or digital means may also be employed. A motorized pump and digital display may be used. The valve may include digital or electric means to change or modify pressure at a set rate or intervals or based on feedback from the monitoring means. The system may include various sensors and monitors. Other sensors could be time set for instance if the system was rented so that the system would stop working once the sensor triggered that the rental days or rental hours had expired. In this instance, the sensor could be reset if additional rental time was purchased.

The inventive therapeutic compression system may be used for post-hemodialysis treatment to control or reduce external bleeding and swelling at the needle access site. In use the therapeutic compression apparatus 100, 200, 300 may be placed by the patient, practioner or care-giver on the chosen limb, such as for instance the lower arm or upper arm or wrist or two apparatus may be employed one on each of the lower arm and the upper arm or wrist at the same time depending on where the needle access sites are located on the patient. The therapeutic compression apparatus 100, 200, 300 are fastened around the arm area of the patient over the needle access site, absorbent dressing, and needle itself. The patient, practitioner or care-giver fastens or secures the fastening tabs up or down to the arm. If there are additional optional straps located on the proximal end of the apparatus the first strap should be closed or secured in a tight fashion so that the therapeutic compression apparatus 100, 200, 300 fits snugly but not too tight and the second strap should be closed or secured in a tight fashion so that the therapeutic compression apparatus 100, 200, 300 fits snugly but not too tight. The patient, practioner or care-giver then removes the cap 113, 213, 313 from the inflation port 112, 212, 312 located on the therapeutic compression apparatus 100, 200, 300 thus opening, namely the female slip luer housing 214. The patient, practioner or care-giver then selects a pressure amount of value on the dial of the inflation means 150 depending on the treatment and whether the patient or if no dial provided starts the inflation means, such as squeezing the hand bulb 420. Once pressure amount or value is chosen on the dial (or set to one pressure such as 60 mm-Hg where there is no dial and only one given pressure amount), the corresponding umbrella valve or switch is activated such that the pressure is thereafter maintained (closed position) or modified so as to maintain the pressure as it changes with the activity or altitude when in use). The patient, practioner or care-giver then inserts an end of the tube 160 portion such as the male luer slip 161 into the inflation port 112, 212, 312 on the therapeutic compression apparatus 100, 200 300, uses the hand pump 420, and the air or fluid is increased to inflate the bladder 102, 202, 302 and thus achieve a desired pressure amount or valve. Again this inflation means may be a hand pump, electric pump, battery-operated pump, remote controlled pump, airpump, gas pump, or any other known inflation means. A number or variety of inflation means can be employed such as a manual pump, hand pump, foot pump, mechanical pump, electrical pump, battery-operated pump, static pump, intermittent pump, varying pump, automatic pump, pneumatic pump, negative pressure pump, suction pump or vacuum, pulsing pump, or any other known or developed source of inflation so as to provide a celtain pressure within the bladder so to provide compression in use by the patient. Depending on the inflation means employed such inflation means may be removed and the cap 113, 213, 313 replaced and the pressure will not decrease except as noted in position or the inflation port may be self-sealing as shown in FIGS. 16A-B. At any point in use the patient, practioner or care-giver can deflate the bladder by either inserting the cap 113, 213, 313 so it depresses the valve spring and thus release the air or fluid in the bladder 102, 202, 302 and decrease the pressure, or the patient, practioner or care-giver can reinsert the inflation means and select the “Deflate” or “Release” on the dial if so provided and the corresponding umbrella valve will be in the open position so as to release the air or fluid in the bladder and decrease the pressure until a deflated state is achieved for the bladder and the therapeutic compression apparatus. In another embodiment a release button 130, 230, 330 configuration is within the air bladder 102, 202, 302 as shown in FIGS. 4-5 and 10-11 which can immediately release the pressure within the air bladder 102, 202, 302. The therapeutic compression apparatus can be reinflated and deflated over and over again when in use.

The method of use of the inventive system has been illustrated and described with respect to specific embodiments thereof, which embodiments are exemplary and illustrative of the principles of the invention and are not intended to be exclusive or otherwise limiting embodiments. For instance, while in the foregoing embodiments the therapeutic compression apparatus 100, 200, 300 are described as having inflatable bladders, the therapeutic compression apparatus 100, 200, 300 may additionally include integrally formed or attached (e.g., by adhesive. radio-frequency welding, etc.) compression members that are not configured for inflation and/or deflation. For instance, additional compression members may be implemented using any of a variety of preformed and/or prefilled cushioning materials such as foam cushions and/or air, gel, or other fluid filled non-inflatable cushions, provided such compression members generate sufficient compression in combination with integral compression bladders. Further, while particular shapes, sizes, and materials have been described for purposes of illustration, it will be recognized that any of a variety of shape or size can be used, and the materials described are not exclusive but merely illustrative. Also, as noted above, while the bladder shown is inflated with air, it will be appreciated that any other fluid or medium such as liquid or gel can be used. Moreover, as also noted, it will be understood that bladders may be configured to have multiple pneumatically independent and/or pneumatically coupled bladder sections, and may also be configured to have various contours or lobulations. The inventive therapeutic compression system described herein can be used for any suitable condition treatable by compression therapy and the like. For example, the inventive system including a therapeutic compression apparatus 100, 200, 300 in accordance with the present invention can be used for compression of the venous system for the treatment of swelling and bleeding post-hemodialysis treatment and the like.

As stated herein, the possible therapeutic compression apparatus to be used in the inventive system are only limited to the target limbs or body parts to be compressed or subject to pressure treatments in order to reduce and treat swelling and bleeding (even including lymphedema, CVI, DVT or any other medical issue). For instance, the therapeutic compression apparatus could be for a foot, ankle, calf, lower leg, knee, thigh, groin, hip, buttocks, torso, stomach, back, shoulder, chest, arm, elbow, wrist, hand, neck, head, or the like and any combinations thereof. The inventive system of the instant invention described herein solves many problems with the prior art and in the industry and treatment of patients.

The therapeutic compression apparatus 100, 200, 300 may be applied on the patient's body part by the patient without the need or requirement of a skilled care-giver as required by current devices and apparatus. It further is capable of maintaining sufficient effective pressure without overpressure complications, maintaining compression and the like. The inventive therapeutic compression apparatus controls external bleeding at the needle access site post-dialysis treatment with consistent and constant compression and pressure that is more reliable than manual compression post-dialysis at a needle access site. The medical staff such as nurses can do other tasks and the patient has ease of mind by having visualization of the needle access site to monitor for external bleeding. The patient using the inventive therapeutic compression system and apparatus also has mobility since a medical staff doesn't have to hold down a dressing at the needle access site while the patient sits in a chair. Further the inventive therapeutic compression system and apparatus doesn't slip or move based on the extended compression beyond just the needle access site to a portion of the arm adjacent to the needle access site. The pressure however from the inventive therapeutic compression apparatus is just what is needed to stop or control bleeding and not too high to cause bruising or other complications at the needle access site or on the arm itself.

The inventive therapeutic compression system also reduces the problem of lack of mobility in that the inflation means is not integral to the therapeutic compression apparatus and instead the patient can walk about and go to work, school, recreational activities while the external bleeding and swelling at the needle access site is reduced or eliminated. The compression is held constant with the inventive therapeutic compression apparatus versus a human being applying pressure, such as a medical professional, by pressing down with a finger or hand on the needle access site once the needle is removed from the patient's arm and the pressure varies as the medical professional gets tired from pressing down. The inventive system includes an inflation means which is less bulky than known systems and does not require a human being trained professional medical staff to manually apply pressure and compression to the needle access site after hemodialysis treatment. The inventive system and apparatus can be used by the patient in between treatment sessions and thus the user can return to life activities sooner than with known compression systems, prophylaxis systems and other treatment systems which limit the user's ambulation both within and outside the home due to power constraints (electrical, mechanical, battery, manual, etc.) on the system. The inventive therapeutic compression apparatus is also compact and smaller being able to fit under clothes on the patient and out of public view. However, the transparent portion 104, 204, 304 of the bladder allows visualization of the needle access site when in view. Further, the inventive system may include one or more sensors to measure the user's limb in regard to pressure on the skin, motion of the limb, blood pressure, tannometer sensor, GPS sensor, and the like while the system is in use. Such sensors may be connected to the inflation means so as to regulate the pressure from the inflation means and either increase or decrease the current pressure level(s). Such sensors may also be connected to a database and possibly accessible to a medical professional and/or the user in real time or as saved over time.

The inventive therapeutic compression apparatus 100, 200, 300 is reusable thereby reducing costs to a dialysis center and to the patient, as well as the inflation means 150 being reusable. Additionally the system incudes multiple sponges or foam or prefabricated air bladders (PFAB as disclosed in Applicant's co-owned patents and pending applications) 460 with optional adhesive to adhere the PFAB to the user's skin, which may be disposable or reusable depending on the materials. Additional gauze or bandages 500a, 500b, 450 may also be included in the system or kit.

The safety features of the inventive therapeutic compression system and apparatus include check valves so ensure pressure and compression is not too high causing complications at the needle access site post-dialysis treatment. For example, the inflation means as shown in FIGS. 14A-C and 15A-G include an umbrella valve 185 chosen for height and durometer of material, such as silicone or other polymers or plastics, based on the pre-determined interface pressure selected as when this blow off pressure is exceeded the umbrella valve 185 flexes and directs excess pressure out of the vent slot 183 (or slots 183a, 183b, 183c, 183d, etc.) and therefore the pre-determined maximum interface pressure for the needle access site is not exceeded which if exceeded could cause complications based on impediment of blood flow through the vessels under the skin. Therefore, the inventive therapeutic compression system decreases complications following dialysis treatment at the needle access site and surrounding the needle access site which complications from compression include cutting off blood circulation, compartment syndrome, thrombosis, aneurism, or even necrosis.

Another embodiment of the present invention not shown includes an assembly according to the invention includes a pressure mechanism having a flexible member for attachment to a limb and an air chamber which may be pumped up into a desired pressurized state, a separate relatively small pre-filled air bladder, an absorbent foam, sponge or dressing coupled to the pre-filled air bladder. Optionally a suction conduit may be coupled to a source of negative pressure (suction) and in fluid communication with the absorbent foam, sponge or dressing. In a preferred embodiment, the pre-filled air bladder (PFAB), the absorbent foam, sponge or dressing and the suction conduit are formed together as a unit. According to one aspect of the invention, the flexible member of the pressure mechanism is adapted to wrap around an arm and over the pre-filled air bladder in order to secure the pre-filled air bladder and the foam, sponge or dressing to a wound or ulcer in the extremity. Thus, the flexible member is provided with some fixation structure such as a hook and loop closure mechanism. An air pumping mechanism is preferably coupled to the air chamber of the pressure mechanism in order to inflate the air chamber to a pressurized state. The air chamber of the pressure mechanism is preferably designed to apply pressure along a predefined area (e.g., the lower arm or upper arm over the needle access site) as opposed to around an entire limb.

According to another aspect of the invention not shown, the suction conduit is located either between the pre-filled air bladder PFAB 460 and the absorbent foam, sponge or dressing which is adhered to the small air bladder, or the pre-filled air bladder PFAB 460 is formed as a donut with a central opening and the suction conduit extends through the central opening. By coupling the suction conduit to a source of negative pressure, exudate from the bleeding needle access site is sucked through the foam, sponge or dressing into the suction conduit. One of the methods of the invention not shown include locating the pre-filled air bladder PFAB 460 and foam, sponge or dressing over a bleeding needle access site on an arm 600, wrapping the flexible member of the pressure mechanism around a limb with the air chamber located over the pre-filled air bladder/absorbent foam, sponge or dressing, and fastening the pneumatic pressure mechanism in place with the fixation structure. When the apparatus is properly located and affixed to the lower arm or upper arm of two wrap located on each of the lower arm and upper arm at the same time, the air chamber is inflated, preferably to 30-40 mm Hg, thereby applying pressure to the limb and more specifically via the pre-filled air bladder to the wound. The suction apparatus is activated by turning on the source of negative pressure, and exudate from the needle access site is pulled through the absorbent foam, sponge or dressing into the suction conduit.

Other embodiments for the present invention are not shown but have the advantages of reducing and/or eliminating external bleeding at the needle access site both immediately after the hemodialysis treatment or for a time in between treatment sessions. The patient can apply the inventive therapeutic compression apparatus 100, 200, 300, 350 by himself or herself without the aid or time spent by a trained medical professional staff such as a dialysis technician or nurse. The inventive therapeutic compressions apparatus 100, 200, 300, 350 includes a translucent or transparent bladder portion 104, 204, 304 allowing for visual observation for any external bleeding occurring at the needle access site once the needle is removed following the hemodialysis treatment. The compression or pressure level remains constant to reduce or eliminate bleeding and swelling on the arm, both at or near the needle access site. Other advantages are found through the inventive system, apparatus, method and kit.

While the subject invention of the present disclosure has been described with respect to preferred and exemplary embodiments, those skilled in the art will readily appreciate that various changes and/or modifications can be made to the invention without departing from the spirit or scope of the invention as described herein. There have been described and illustrated herein several embodiments of an intermittent pressure apparatus and a method of installing and operating same. While particular embodiments of the invention have been described, it is not intended that the invention be limited thereto, as it is intended that the invention be as broad in scope as the art will allow and that the specification be read likewise. Thus, while particular shapes and sizes of inflatable bladders, transparent or translucent or cleai1 portions of the air bladder, and straps have been disclosed, it will be appreciated that other shapes, sizes, and attachment means may be used as well. It will also be understood that while Velcro and adhesive means have been disclosed for helping to secure the therapeutic compression apparatus 100, 200, 300, 350 to the arm, other types of attachments such as hooks, snaps, or wraps may be used. In addition, it will be appreciated that while the fluid conduit may be detachably connected to the bladders using mating threaded portions or bayonet locks, other means of attachment known in the art may be used. It will therefore be appreciated by those skilled in the art that yet other modifications could be made to the provided invention without deviating from its spirit and scope as claimed.

We claim:

1. An arm therapeutic compression apparatus for post-dialysis treatment of a patient comprising:
a wrap having an inner sheet and an outer sheet joined together by welds and lines to create a bladder;
the bladder including a transparent portion configured to be worn over a needle access site on an arm of a user allowing visualization of the needle access site;
the wrap including a securing means to secure the wrap to the user's arm wherein a portion of the securing means is elongated and configured to allow the user to self-apply the wrap when in use; and
an inflation port connected to the bladder configured to be accessible to both static and intermittent inflation means.

2. The therapeutic compression system of claim 1 wherein the inflation means is selected from the group consisting of manual pumps, static pumps, intermittent pumps, electrical inflation pumps, battery inflation pumps, gas powered inflation pumps, static pneumatic compression pumps, intermittent pneumatic inflation pumps, and the combinations thereof and the inflation means for the at least one bladder is selected from the group consisting of air, gas, fluid or combinations thereof.

3. The therapeutic compression system of claim 1 wherein the inflation means incudes a manual pump including two check valves and configured to provide static inflation at a pressure of about 40 mm-Hg to about 70 mm-Hg.

4. The therapeutic compression system of claim 1 wherein the inflation port is a self-sealing inflation port configured to prevent deflation of the at least one bladder and includes a check valve.

5. The therapeutic compression system of claim 1 further comprising an elbow connector, a check valve and a valve cap wherein the valve cap is capable of releasing a pressure created by the inflation means within the at least one bladder.

6. The therapeutic compression system of claim 1 further comprising a pressure sensor operatively connected to the inflation means to protect from over inflation and wherein the check valve is set to open at a predetermined pressure or a user selectable pressure.

7. The therapeutic compression system of claim 1 wherein the wrap member further comprises a release valve or button for release of pressure within the inflated bladder.

8. The therapeutic compression system of claim 1, wherein the bladder is an air bladder configured to provide compression at the needle access site and a portion of the user's arm.

9. The therapeutic compression system of claim 1, wherein the inflation means includes a real-time pressure measurement mechanism.

10. The therapeutic compression system of claim 1, wherein the inflation means includes a pulsometer configured to measure the pulse of a target vessel in the area of the needle access site.

11. The therapeutic compression system of claim 1 wherein the transparent portion of the bladder is in the shape of a circle, cross, oval, kidney, elongated kidney, square, rectangle, “U”, “C” or the like shapes.

12. The therapeutic compression system of claim 1 further comprising an absorbent dressing selected from the group of bandages, gauze, foam, PFAB and the like.

13. The therapeutic compression system of claim 12 wherein the absorbent dressing is a foam disk including a center opening and a slit on the radius of the disk configured to accommodate a needle at the needle access site.

14. The therapeutic compression system of claim 1 further comprising an umbrella valve within the inflation means configured to prevent overinflation of the bladder on the needle access site.

15. The therapeutic compression system of claim 1, wherein the wrap includes an inner sheet of wicking material and an outer sheet partially elastic or having an elastic portion configured to securely wrap around the arm of the user prior to and after inflation of the bladder.

16. A method of applying compression pressure to a needle access site on the arm of a user after dialysis treatment, comprising:
placing a dressing over a needle and needle access site on the arm of a user;
placing a wrap on an arm of a user, wherein the at least one wrap includes (a) a bladder connected to an inflation means with the bladder having a transparent portion configured to allow visualization of the needle access site when a needle is in the arm of the user and (b) an elongated securing means configured to accommodate a temporary adhesive for self-application and securing of the wrap by the user without assistance;
securing the wrap on the arm with the transparent portion of the bladder over the needle and needle access site on the arm of the user;
connecting an inflation means to an inflation port on the wrap;
activating the inflation means and the bladder;
removing the needle from the user leaving an absorbent dressing on the needle access site; and
monitoring bleeding at the needle access site via the transparent portion of the bladder.

17. The method of claim 16 further including the step of deflating the inflated bladder by opening a release valve or button on the wrap and releasing a pressure within the at least one bladder.

18. A therapeutic compression system configured to control external bleeding at a needle access site on an arm of a patient post-dialysis treatment, comprising:
at least one wrap having a proximal end configured to be located along a wrist, elbow, shoulder or arm of a user and a distal end of the primary wrap configured to be located at a wrist, elbow, shoulder or arm of a user, the primary wrap including at least one bladder;
a secondary wrap having a proximal end and a distal end wherein the proximal end of the secondary wrap is connected to the distal end of the primary wrap;
the at least one bladder having a translucent or transparent portion configured for visualization of a needle access site on the user;
an inflation means connected to the bladder via a universal inflation port on the at least one bladder wherein the inflation port includes a check valve and is configured to be capable of connection to a manual or powered pump, and
an assembly comprising: a) pressure mechanism having a flexible member for attachment around a limb and an air chamber which assumes a first pressurized stated, said air chamber having a length and a width, said width being less than half the at least one bladder; b) a pre-filled air bladder having a length and a width smaller than the width of the air chamber; c) an absorbent foam, sponge or dressing coupled to the pre-filled air bladder; and d) a suction conduit in fluid communication with the absorbent foam, sponge or dressing and adapted for coupling to a source of negative pressure.
